(19) United States
(12) Patent Application Publication
(10) Pub. No.: US 2023/0172990 A1
(43) Pub. Date: Jun. 8, 2023

(54) EXTRACELLULAR VESICLE COMPOSITIONS AND THE USE THEREOF IN THE TREATMENT OF SKIN CONDITIONS AND IN IMMUNE MODULATION

(71) Applicant: MVex Japan, Inc., Tokyo (JP)

(72) Inventors: Osamu Ohneda, Tsukuba City (JP); Khanh Cat Vuong, Tsukuba City (JP)

(21) Appl. No.: 17/899,151

(22) Filed: Aug. 30, 2022

Related U.S. Application Data
(63) Continuation of application No. PCT/JP2021/010714, filed on Mar. 5, 2021.
(60) Provisional application No. 63/124,598, filed on Dec. 11, 2020.
(60) Provisional application No. 63/019,252, filed on May 1, 2020.
(60) Provisional application No. 62/985,749, filed on Mar. 5, 2020.

(51) Int. Cl. A61K 35/28; A61P 17/02; A61P 11/00

(52) U.S. Cl. CPC A61K 35/28; A61P 17/02; A61P 11/00

(57) ABSTRACT

Extracellular vesicle (EV) compositions, specifically exosomes and microvesicles are disclosed together with the use of such EV compositions, exosomes and microvesicles, in treatment of skin disorders, and lung conditions such as COVID-19, over-reactive inflammatory responses, cytokine storms and/or ARDS.

### CROSS-REFERENCE TO RELATED APPLICATIONS

This Patent Application is a continuation of International Application No. PCT/JP2021/010714, filed Mar. 5, 2021, which claims priority to U.S. Provisional Patent Application No. 62/985,749, filed Mar. 5, 2020, entitled, “Extracellular Vesicle Compositions and the Use Thereof in the Treatment of Skin Conditions and Cancer”, U.S. Provisional Patent Application No. 63/019,252, filed May 1, 2020, entitled, “Extracellular Vesicle Compositions and the Use Thereof in the Treatment Of Skin Conditions, Cancer, Pneumonia, Cytokine Storms, ARDS, and COVID-19”, and U.S. Provisional Patent Application No. 63/124,598, filed Dec. 11, 2020, entitled, “Extracellular Vesicle Compositions and the Use Thereof in Immune Modulation”, all of which are incorporated herein by reference in their entirety.

### TECHNICAL FIELD

The disclosure relates to extracellular vesicle compositions, which include exosomes and microvesicles, and the use thereof in treatment of skin disorders, and lung conditions such as COVID-19, over-reactive inflammatory responses, cytokine storms and/or ARDS.

### BACKGROUND

Mesenchymal stem cells (“MSCs”) are progenitor cells having the capacity to differentiate into neuronal cells, adipocytes, chondrocytes, osteoblasts, myocytes, cardiac tissue, and other endothelial and epithelial cells. MSCs may be defined phenotypically by gene or protein expression or by function and may be obtained from a number of sources including but not limited to bone marrow, blood, periosteum, dermis, umbilical cord blood, Wharton's Jelly, and placenta.

MSCs can be divided into, (1) adult and (2) fetal/perinatal MSCs, derived from (1) adult bone marrow (BM-MSCs), adipose tissue (AT-MSCs) (adult/elderly or infant), or (2) from fetal/perinatal tissues, including cells obtained from the embryo/fetus itself and cells obtained from extra-embryonic tissues such as placenta, umbilical cord, Wharton's jelly mesenchymal stem cells (WJ-MSCs) and amniotic membranes (Marino L, et al., Int J Stem Cells. 12(2): 218-226, 2019). MSCs isolated from adult tissues have a very limited proliferative capacity, while MSCs derived from infant and extra embryonic tissues exhibit more potential for therapeutic uses.

Wharton's jelly mesenchymal stem cells (WJ-MSCs) have a high differentiative potential. WJ-MSCs have immunomodulatory properties and express surface markers, in accordance with the commonly accepted minimal criteria of the International Society for Cellular Therapy (ISCT), such as CD90, CD105, CD44, CD73, CD9, and very low levels of CD80 (Marino L, et al., Int J Stem Cells. 12(2): 218-226, 2019).

MSCs are known for their anti-inflammatory effects, wound healing, and immunoregulatory effects generally mediated in a non-contact fashion. MSCs have been the subject of preclinical and clinical studies, including acute myocardial infarction, stroke, acute kidney failure, and many others. (See, http://clinicaltrials.gov.)

MSCs have been demonstrated to have immunomodulatory functions and anti-inflammatory activity and have been suggested for therapeutic treatment of various diseases. (Song N. et al. (2020) Trends Pharmacol Sci 41: 653-664).

Extracellular vesicles (EVs) are released by cells, such as MSC, and have been identified as having a role in cell-to-cell communication. The content of EVs includes lipids, nucleic acids, and proteins, specifically proteins associated with the plasma membrane, cytosol, and those involved in lipid metabolism. EVs transfer such molecules between adjacent cells and to distant cells via the circulation. The molecules transferred by the EVs are determined by the parent cell and play a fundamental biological role in the regulation of normal physiological as well as pathological processes. EVs are stable in circulation and have low immunogenicity and toxicity.

EVs are small membrane vesicles with a diameter of 20 nm to 2 μm that are bounded by a phospholipid bilayer and released by all cell types in various biological fluids and extracellular spaces. EVs can be classified into different subpopulations, including apoptotic bodies (ABs), microvesicles (MVs) and exosomes, each with specific characteristics (Zaborowski, M. P., et al., Bioscience, 65, 783-797, 2015). EVs contain surface receptors, membrane and soluble proteins, lipids, RNAs (e.g., mRNA, microRNA, tRNA, rRNA, small nucleolar RNA, small circular nucleolar RNA, piRNA, scaRNA, viral RNA, Y RNA, and long noncoding RNA), and have also been reported to contain genomic and mitochondrial DNAs (Yu, Maria et al., BioMed Research International, Volume 2018, 27 pages, Article ID 8545347). EVs can package proteins, nucleic acids and lipids, and deliver them to another cell, neighboring or distant, and thereby alter the recipient cell's functions.

Apoptotic bodies are vesicle-like structures that form as a result of cell fragmentation in the process of programmed cell death (apoptosis). Apoptotic bodies range in size from approximately 500 to 2000 nm and are characterized by the presence of DNA fragments and histones along with proteins.

MVs originate directly from cell membranes through outward budding of the cell's plasma membrane. They have a diameter that is typically from 100 to 1000 nm, and they are characterized by the presence of phosphatidylserine (PS) in their outer membrane. Because MVs form by an outward budding of the cell's plasma membrane, MVs contain mainly cytosolic and plasma membrane associated proteins, in particular, proteins known to cluster at the plasma membrane surface, with the inner contents of MVs mirroring that of their parent cells. MVs, like exosomes are involved in communication between local and distant cells. MVs expose phosphatidylserine (PS) on the outer leaflet of the membrane and when stained with Annexin-V, they can be identified by flow cytometry. In addition, CD40 ligand, ADP-ribosylation factor 6 (ARF6), and different proteins associated with lipid rafts, such as integrins and flotillins have been reported as MV markers. (Chiriacó MS, et al. Sensors (Basel) 18(10): 3175, 2018).

Exosomes are currently characterized as having a diameter of less than 100 nm, typically 20 to 100 nm. Exosomes originate from invagination of the lipid bilayer membrane of multivesicular bodies, and at any given point, they may contain known molecular constituents of a cell, including proteins, RNA, and DNA that mirror their parent cells (Samanta, Saheli et al., Acta Pharmacologica Sinica, 39: 501-513, 2018). Exosomes participate in cell to cell communication, cell maintenance, tumor progression, and can present antigens. In the nervous system, exosomes have been shown to play a role in tissue repair and regeneration (Doyle, L M, and Wang, MZ, Cells, 8, 727, 2019). Exosomal membranes contain several endosome-specific proteins, including TSG101, Alix, and the tetraspanins CD9, CD63, and CD81 (Chiriacó MS, et al. Sensors (Basel). 18(10): 3175, 2018).

The present disclosure provides EV compositions, exosomes and MVs, and methods of their use in the treatment of skin disorders, pneumonia, ARDS, cytokine storms, and COVID-19.

The incidence of skin conditions such as diabetic and pressure ulcers is increasing due to the aging population and the management of such conditions is increasingly important.

The difficulty in healing cutaneous wounds and ulcers is a significant health problem that impacts the quality of life for millions of people. It has been suggested that stem cells and progenitor cells can promote wound healing and that secreted factors associated with extracellular vesicles (EVs) contribute to observed therapeutic benefits.

Stem cell-based therapies using MSCs, embryonic stem cells, and pluripotent stem cells (PSCs) for the treatment of skin conditions such as pressure ulcers have shown potential for therapeutic efficacy in preclinical and early clinical studies (Heublein H, et al., Drug Discovery Today, 20: 703-17, 2015). However, stem cell therapy presents significant regulatory and technical challenges. Local administration of EVs at wound sites has demonstrated accelerated wound healing in animal models of cutaneous wounds. (Ren, S. et al., Stem Cell Res. Ther., 10, 47, 2019).

EVs appear to have the potential to play an important role in future therapeutic approaches to treatment of skin conditions and a variety disease conditions.

Severe acute respiratory syndrome coronavirus 2 (SARS-CoV-2), the causative agent of COVID-19 was first reported in Wuhan, China in December 2019. Since the initial cases of COVID-19 were reported SARS-CoV-2 has emerged as a global pandemic with an ever-increasing number of severe cases that threaten to overwhelm health care systems in many parts of the world (Huang, C. et al. (2020) Lancet 395, 497-506.

To date, there is no cure for COVID-19, and clinical management of patients currently is focused on treatment with corticosteroids such as Dexamethasone, antibody therapy, an antiviral medications such as remdesivir, and supportive care, including supplemental oxygen and mechanical ventilation support in some cases.

Metcalfe, S. M. (2020). Medicine in Drug Discovery, 100019; Golchin, A. et al., (2020) Stem Cell Reviews and Reports, April 13: 1-7), however, despite large numbers of clinical trials, there are no proven treatments for COVID-19

The S protein of SARS-CoV-2 binds to receptors including Angiotensin-Converting Enzyme 2 (ACE2) and enters cells in a manner catalyzed by transmembrane protease serine 2 (TMPRSS2) protease. In lung epithelial cells, SARS-CoV-2 infection activates the release of interferon, resulting in the recruiting of monocytes, and activation of monocyte-derived inflammatory macrophages, and caspase I which results in the releases of pro-inflammatory cytokines (Merad M. and JC Martin (2020) Nat Rev Immunol 20: 355-362; Liu Q, et al. (2016) Cell Mol Immunol 13: 3-10).

The enhanced release of pro-inflammatory cytokines can cause a cytokine storm which impairs the function of organs and can causes the death of patients (Bhaskar S et al. (2020) The REPROGRAM Consortium Position Paper. Front Immunol 11: 1648).

Numerous reports showed that the patients with diabetes have an increased risk of severe illness from SARS-CoV-2 infection. A recent meta-analysis of 16 published studies with 3994 patients, conducted by Nandy, K. et al. (2020) Diabetes Metab Syndr. 14(5): 1017-1025, showed that diabetic patients had 3.07-fold higher chances of severe events which were statistically significant, and the presence of Diabetes mellitus had a statistically significant impact on death in COVID 19 patients. In addition, a recent study suggests that together with the cytokine storm induced by SARS-CoV-2 infection, the chronic inflammation in patients with diabetes might contribute to the more severe outcome of dysregulation of inflammatory reaction in COVID-19 (Apicella, M. et al, (2020) Lancet Diabetes Endocrinol 8: 782-792).

In a clinical study in China, MSC were transplanted into seven COVID-19 patients and it was reported that after 2 to 4 days of transplantation, pulmonary function and symptoms of the seven patients were significantly improved, and the activation of cytokine-release immune cells was decreased (Leng Z. et al. (2020) Aging Dis 11: 216-228).

In the US, it is hypothesized that MSCs could reduce the acute lung injury and inhibit the cell-mediated inflammatory response induced by SARS-CoV-2, however, as of December 2020, the National Institute of Health (NIH) COVID-19 Treatment Guidelines Panel recommended against the use of mesenchymal stem cells for the treatment of COVID-19, except in a clinical trial.

There is a need for further evaluation of the efficacy of MSCs and the mediators of observed immunomodulatory functions and anti-inflammatory activity. The present disclosure addresses this need.

### SUMMARY

An extracellular vesicle (EV) composition comprising microvesicles (MVs) and exosomes is provided wherein the composition is derived from mesenchymal stem cells (MSCs), for example MSCs derived from infant adipose tissue, or MSCs derived from Wharton's jelly.

The EV composition may comprises exosomes with a diameter of less than 100 nm and microvesicles (MVs) with a diameter of from about 100 nm to about 1000 nm.

The exosomes and MVs may express CD63 and/or TSG101 membrane proteins.

The EV composition may be derived from MSCs that have been cultured in the presence of Edaravone.

The EV composition may comprise exosomes and MVs in a ratio of from about 0.8:1 to about 1:1.3, from about 1:1.3 to about 0.8:1, from about 1:1, 1:1.5, 1:2, 1:2.5, 1:3, 3:1, 2.5:1, or 1.5 to 1.

The EV composition may be used to treat skin disorders or skin wounds, for example skin disorders or skin wounds that result in necrosis, wherein the necrotic area associated with the skin disorder or skin wound is decreased following treatment with an EV composition comprising a mixture of exosomes and MVs.

The skin disorder or skin wound may be a diabetic ulcer (e.g. from a patient with Type 2 diabetes), a pressure wound such as a bed sore, or an acute wound such as a burn.

Following treatment of a necrotic skin wound or disorder by application of a disclosed EV composition, the necrotic area associated with the skin wound or disorder may be decreased by at least 10%, at least 20%, at least 30%, at least 40%, at least 50%, at least 60%, at least 70%, or at least 80% or more within five to fourteen days following treatment.

Also provided is a method of reducing pro-inflammatory cytokine expression in lung cells that was increased following contact with SARS-CoV-2, by administering an EV composition derived from human mesenchymal stem cells (MSCs) and comprising exosomes and MVs, to the lung cells, wherein following administration, pro-inflammatory cytokine expression in the lung cells is significantly reduced.

The pro-inflammatory cytokines may include one or more of TNFα, IL6, IFN1β, IFNγ3, IP-10, and CXCL9.

Further provided is a method of reversing the reduction in CCL17 expression in lung cells that was increased following contact with SARS-CoV-2, by administering an EV composition derived from human mesenchymal stem cells (MSCs) and comprising exosomes and MVs, to the lung cells, wherein following administration the CCL17 expression in lung cells is restored to substantially the same level detected prior to contact with SARS-CoV-2.

The lung cells may be derived from a patient with Type 2 diabetes.

### BRIEF DESCRIPTION OF THE DRAWINGS

FIG. 1A is a graphic depiction of the reactive oxygen species (ROS) expression in elderly-derived AT-MSCs (black bar) compared to infant-derived AT-MSCs (unshaded bar).

FIG. 1B is an image of the results of a Western blot analysis showing expression of antioxidant gene products SOD1, SOD2, SOD3, Catalase, and GPX1., in elderly-derived AT-MSCs compared to infant-derived ATMSCs infant-derived AT-MSCs. Lamin B is included as a loading control.

FIG. 2A is a graphic depiction of mRNA expression for proinflammatory cytokines (IL6, IL8), and chemokines (CCL5, CCL3) in elderly-derived AT-MSCs (black bar) compared to infant-derived AT-MSCs (unshaded bar).

FIG. 2B is a graphic depiction of mRNA expression of growth factors responsible for homing (SDF1) and angiogenesis (VEGF, Ang1, bFGF) in elderly-derived AT-MSCs (black bar) compared to infant-derived AT-MSCs (unshaded bar).

FIG. 3A is a graphic depiction of the effect of antioxidants Edaravone (Eda), N-acetylcysteine (NAC), and Ascorbic Acid (AA) on ROS levels in elderly-derived AT-MSCs, as measured by fluorescence at 495 nm and 525 nm.

FIG. 3B is a graphic depiction of the effect of Eda, NAC, and AA on ROS levels in elderly-derived AT-MSCs after 1 and 6 passages in culture as measured by fluorescence at 495 nm and 525 nm.

FIG. 3C is a graphic depiction of the effect of Eda, NAC, and AA on cellular senescence in elderly-derived AT-MSCs following 6 or 12 passages in culture as measured by (SA) p-galactosidase activity.

FIG. 4A provides images showing the results of an evaluation of the anti-oxidative agents Eda, NAC and AA on the wound healing ability of elderly AT-MSCs in the in vivo streptozotocin-induced diabetic mouse punch biopsy model.

FIG. 4B is a graphic depiction of necrotic area as a measure of wound healing ability in the in vivo streptozotocin-induced mouse punch biopsy model for a study on the effect of the anti-oxidative agents Eda, NAC and AA on the wound healing ability of elderly AT-MSCs.

FIG. 5A is an image of the results seven days after transplantation of infant AT-MSC (iMSC) and elderly AT-MSCs (eMSC) in the in vivo ischemic skin flap model.

FIG. 5B is a graphic depiction of the necrotic area evident seven days following transplantation of infant AT-MSC (iMSC) and elderly AT-MSCs (eMSC) in the in vivo ischemic skin flap model.

FIG. 6A is an image of the results of immunohistochemical analysis for CD-31 expression in the skin tissue of mice injected with iMSC- or eMSC.

FIG. 6B is a graphic depiction of the results of immunohistochemical analysis for CD-31 expression in the skin tissue of mice injected with iMSC or eMSC.

FIG. 7A is a graphic depiction of particle size distribution for an extracellular vesicle (EV) composition using a Particle size Analyzer FDLS3000.

FIG. 7B is an image of the results of characterization of an extracellular vesicle (EV) composition by staining with anti-CD63 and anti-TSG101 antibodies and evaluation by Western Blot.

FIG. 8A is a graphic depiction of particle size distribution of an infant extracellular vesicle (iEV) composition using a Particle size Analyzer FDLS3000.

FIG. 8B is a graphic depiction of particle size distribution of an elderly extracellular vesicle (eEV) composition using a Particle size Analyzer FDLS3000.

FIG. 8C is an image of the results of characterization of an iEV and eEV composition by staining with anti-CD63 and anti-TSG101 antibodies and evaluation by Western Blot.

FIG. 9A is a graphic depiction of cell number from a study on the effect of iEVs incorporated into eMSCs by co-culture to promote the proliferation of target eMSCs.

FIG. 9B is a graphic depiction of the doubling time from a study on the effect of iEVs incorporated into eMSCs by co-culture to promote the proliferation of target eMSCs.

FIG. 10 is a graphic depiction of the mRNA expression of angiogenic cytokines by eMSCs with incorporated iEVs.

FIG. 11A is an image of the necrotic area in the in vivo streptozotocin-induced diabetic mouse punch biopsy model where the effect of adding iEVs to eMSCs on wound healing was evaluated.

FIG. 11B is a graphic depiction of the necrotic area as a measure of wound healing in the in vivo streptozotocin-induced diabetic mouse punch biopsy model from a study comparing the effect of iMSCs and eMSCs with incorporated iEVs or eEVs on wound healing.

FIG. 12A is an image of the necrotic area from a study in the in vivo mouse punch biopsy model in the streptozotocin-induced diabetic mice where the wound healing ability of iEV and eEV was compared.

FIG. 12B is a graphic depiction of the necrotic area as a measure of wound healing ability in the in vivo mouse punch biopsy model in the streptozotocin-induced diabetic mice where the wound healing ability of iEV and eEV was compared.

FIG. 12C is an image of the results of staining the skin tissues of mice transplanted with iEVs or eEVs with anti-CD31 in the in vivo mouse punch biopsy model in the streptozotocin-induced diabetic mice as an indicator of neovascularization ability.

FIG. 12D is a graphic depiction of the results of staining the skin tissues of mice transplanted with iEVs or eEVs with anti-CD31 in the in vivo mouse punch biopsy model in the streptozotocin-induced diabetic mice as an indicator of neovascularization ability.

FIG. 13A is an image of the results of an in vitro scratch assay showing the migration ability of normal (n) AT-MSC, diabetic (d) AT-MSCs, and dAT-MSCs treated with nAT-MSC-derived EVs at 0 and 16 hours.

FIG. 13B is a graphic depiction the results of the wound area from an in vitro scratch assay showing the migration of diabetic nAT-MSC, dAT-MSCs, and dAT-MSCs treated with nAT-MSC-derived EVs at 0 and 16 hours.

FIG. 14A is an image of the necrotic area of mouse skin as a measure of wound healing ability of PBS, nAT-MSC, dAT-MSCs, and dAT-MSCs treated with nAT-MSC-derived EVs in the ischemic mouse flap models in C57BL/6 mice.

FIG. 14B is a graphic depiction of necrotic area as a measure of wound healing ability of PBS, nAT-MSC, dAT-MSCs, and dAT-MSCs treated with nAT-MSC-derived EVs in the ischemic mouse flap models in C57BL/6 mice.

FIG. 15, panel A is an image of the necrotic area of ischemic flap C57BL/6 mice injected with iEVs or eEVs.

FIG. 15, panel B is a graphic depiction of the necrotic area of ischemic flap C57BL/6 mice injected with iEVs or eEVs.

FIG. 15, panel C is an image of the results of immunohistochemical staining with anti-CD31 of the necrotic areas of ischemic flap C57BL/6 mice injected with iEVs or eEVs on the seventh day of transplantation.

FIG. 15, panel D is a graphic depiction of the results of immunohistochemical staining with anti-CD31 of the necrotic areas of ischemic flap C57BL/6 mice injected with iEVs or eEVs on the seventh day of transplantation.

FIG. 15, panel E is a graphic depiction of the survival rate of db/db mice injected with iEVs or eEVs.

FIG. 15, panel F is an image of the necrotic area of ischemic flap db/db mice injected with iEVs or eEVs on day 2, 3 and 7 post injection.

FIG. 16A is a graphic depiction of the proliferative ability of Wharton Jelly MSCs (WJ MSCs) as compared to infant AT-MSCs.

FIG. 16B is a graphic depiction of mRNA expression of angiogenesis related genes, including vegf, fgf, pdgf-bb, and sdf-1 in WJ MSC as compared to infant AT-MSCs.

FIG. 17A is an image of the necrotic area of in vivo streptozotocin-induced diabetic mouse punch biopsy model injected with PBS, EVs derived from infant AT-MSCs, or WJ MSCs.

FIG. 17B is a graphic depiction of the necrotic area of in vivo streptozotocin-induced diabetic mouse punch biopsy model injected with PBS, EVs derived from infant AT-MSCs, or WJ MSCs.

FIG. 17C is an image of the results of immunohistochemical analysis for CD-31 expression in the necrotic area of in vivo streptozotocin-induced diabetic mouse punch biopsy model injected with PBS, EVs derived from infant AT-MSCs, or WJ MSCs.

FIG. 17D is a graphic depiction of the results of immunohistochemical analysis for CD-31 expression in the necrotic area of in vivo streptozotocin-induced diabetic mouse punch biopsy model injected with PBS, EVs derived from infant AT-MSCs, or WJ MSCs.

FIG. 18A is an image of the particle size distribution of microvesicles derived from WJ MSCs.

FIG. 18B is an image of the particle size distribution of exosomes derived from WJ MSCs.

FIG. 19A is an image of the necrotic area in the in vivo mouse punch biopsy model in db/db diabetic mice where the wound healing ability of MVs, exosomes and a mixture of exosomes and MVs was compared, with the amount of MVs or exosomes injected normalized based on μg of protein.

FIG. 19B is a graphic depiction of the necrotic area in the in vivo mouse punch biopsy model in db/db diabetic mice where the wound healing ability of MVs, exosomes (Exo) and a mixture of MVs+exosomes (Exo) was compared, with the amount of MVs or Exos injected normalized based on μg of protein.

FIG. 20A is an image of the necrotic area in the in vivo mouse punch biopsy model in db/db diabetic mice where the wound healing ability of MVs alone, Exos alone, and a mixture of MVs+Exos in a (1:1), (2:1) and (1:2) ratio was evaluated with the amount of MVs or exosomes injected normalized based on μg of protein.

FIG. 20B is a graphic depiction of the necrotic area in the in vivo mouse punch biopsy model in diabetic mice (T2DM db/db) where the wound healing ability of MVs alone, Exos alone and a mixture of MVs+Exos in a (1:1), (2:1) and (1:2) ratios was evaluated with the amount of MVs or Exos injected normalized based on μg of protein.

FIG. 21A is an image of the necrotic area in the in vivo streptozotocin-induced diabetic mouse punch biopsy model where the wound healing ability of 0 μg, 2 μg, 4 μg, and 6 μg, respectively, of WJ-EVs was evaluated.

FIG. 21B is a graphic depiction of the necrotic area in the in vivo streptozotocin-induced diabetic mouse punch biopsy model where the wound healing ability of 0 μg, 2 μg, 4 μg, and 6 μg, respectively, of WJ-EVs, was evaluated.

FIG. 22A is an image of the necrotic area in the in vivo mouse punch biopsy model in streptozotocin-induced diabetic mice where the wound healing ability of 4 μg of WJ-EVs stored for 6 months at −30° C., 1 year at −30° C., and 1 year at −80° C. was evaluated.

FIG. 22B is a graphic depiction of the necrotic area in the in vivo mouse punch biopsy model in streptozotocin-induced diabetic mice where the wound healing ability of 4 μg of WJ-EVs stored for 6 months at −30° C., 1 year at −30° C., and 1 year at −80° C., respectively, was evaluated.

FIG. 23A is a graphic depiction of the survival rate of db/db mice injected with eEVs (elderly AT-MSC-derived EVs), WJ-EVs (Wharton's Jelly MSC-derived EVs), or Edaravone-treated-WJ-EVs.

FIG. 23B is an image of the necrotic area of ischemic flap db/db mice injected with eEVs, WJ-EVs, or Edaravone-treated-WJ-EVs on day 6 post injection.

FIG. 23C is a graphic depiction of the necrotic area of ischemic flap db/db mice injected with eEVs, WJ-EVs, or Edaravone-treated-WJ-EVs on day 6 post injection.

FIG. 24A is a microscopic image showing the morphology of Calu-3 human lung epithelial cells alone (No induction) or following exposure to 30 pmol SARS COV2 spike protein peptides (+Prot_S) for 24 hours (+Prot_S).

FIG. 24B is a graphic depiction of the proliferation of Calu-3 human lung epithelial cells alone (No induction) or following exposure to 30 pmol SARS COV2 spike protein peptides (+Prot_S) for at 48 and 96 hours.

FIG. 24C is a graphic depiction of the doubling time of Calu-3 human lung epithelial cells alone (No induction) or following exposure to 30 pmol SARS COV2 spike protein peptides (+Prot_S) for 24 hours.

FIG. 24D is a graphic depiction of the expression of CCL17 in Calu-3 human lung epithelial cells alone (No induction) or following exposure of Calu-3 human lung epithelial cells to 30 pmol SARS COV2 spike protein peptides for 24 hours (+Prot_S). CCL17 expression was determined by qPCR as described above under the section entitled “Gene Expression Analysis”.

FIG. 24E is a graphic depiction of the expression of pro-inflammatory cytokines (TNFα, IL6, IFN1β, IFNγ3, IP-10, and CXCL9) in Calu-3 human lung epithelial cells alone (No induction) or following exposure to 30 pmol SARS COV2 spike protein peptides (+Prot_S) for 24 hours. Pro-inflammatory cytokine expression was determined by qPCR as described above under the section entitled “Gene Expression Analysis”.

The results in FIGS. 24A-E are presented as the mean±SD. ***P<0.01, **P<0.01, *P<0.05, ns: no significance. The analyses were performed in triplicate.

FIG. 25, panel A is a graphic depiction of the characterization of nAT-MSCs and nWJ-MSCs showing the growth of the MSCs over 10 days indicating a high proliferation rate.

FIG. 25, panel B is an image showing the adipogenic and osteogenic differentiation of nAT-MSCs and nWJ-MSCs after 20 days. Adipo: Adipogenic differentiation, Osteo: Osteogenic differentiation.

FIG. 25, panel C is a graphic depiction of the results of FACS analysis for cell surface expression of MSC markers CD73, CD90, and CD105 (positive), and CD45 and CD31 (negative) on nAT-MSCs and nWJ-MSCs.

FIG. 25, panel D is a graphic depiction of pro-inflammatory cytokine expression in human lung epithelial cells alone (No induction), exposed to SARS COV2 spike protein peptides (+Prot_S), or co-cultured with nAT-MSCs (+Prot_S+nAT-MSCs) or nWJ-MSCs (+Prot_S+nWJ-MSCs), as determined by PCR. Four independent cell lines of MSCs were used in the experiments. n=4.

FIG. 25, panel E is an image showing the expression morphology of n-EVs (derived from nAT-MSCs) and nWJ-EVs (derived from nWJ-MSCs) using transmission electron microscopy.

FIG. 25, panel F is an image showing the results of Western Blot analysis of nAT-MSCs, nWJ-MSCs, n-EVs and nWJ-EVs indicating positive expression of EV markers CD63 and TSG101, and the lack of expression of actin.

FIG. 25, panel G is an image showing the results of fluorescent microscopy of untreated Calu-3 cells (no induction), Calu-3 cells treated with 30 pmol Prot_S for 24 hours (+Prot_S), Calu-3 cells treated with 30 pmol Prot_S for 24 hours and nEVs for an additional 24 hours (+Prot_S+nEVs), and Calu-3 cells treated with 30 pmol Prot_S for 24 hours and nWJ-EVs for an additional 24 hours (+Prot_S+nWJ-EVs). EVs were labeled by PKH-26-red.

FIG. 25, panel H is a graphic depiction of the expression of CCL17 in human lung epithelial cells examined by qPCR showing results for untreated Calu-3 cells (no induction), Calu-3 cells treated with 30 pmol Prot_S for 24 hours (+Prot_S), Calu-3 cells treated with 30 pmol Prot_S for 24 hours and nEVs for an additional 24 hours (+Prot_S+nEVs), and Calu-3 cells treated with 30 pmol Prot_S for 24 hours and nWJ-EVs for an additional 24 hours (+Prot_S+nWJ-EVs).

FIG. 25, panel I is a graphic depiction of pro-inflammatory cytokine expression in human lung epithelial cells examined by qPCR showing results for untreated Calu-3 cells (no induction), Calu-3 cells treated with 30 pmol Prot_S for 24 hours (+Prot_S), Calu-3 cells treated with 30 pmol Prot_S for 24 hours and nEVs for an additional 24 hours (+Prot_S+nEVs), and Calu-3 cells treated with 30 pmol Prot_S for 24 hours and nWJ-EVs for an additional 24 hours (+Prot_S+nWJ-EVs).

For FIG. 25, panels A-I, EVs were isolated from four independent cell lines of MSCs were used in the above experiments. n=4. The data represent the mean±SD. ***P<0.01, **P<0.01, *P<0.05, ns: no significance. The experiments were performed in triplicate.

FIG. 26, panel A is a microscopic image showing the morphology of Calu-3 human lung epithelial cells cultured in the presence of 0, 10 mM, 20 mM or 30 mM glucose for 24 hours.

FIG. 26, panel B is a graphic depiction of the expression of pro-inflammatory cytokines (IL-6, TNFα and IFN-β) in Calu-3 human lung epithelial cells cultured in the presence of 0, 10 mM, 20 mM or 30 mM glucose for 24 hours.

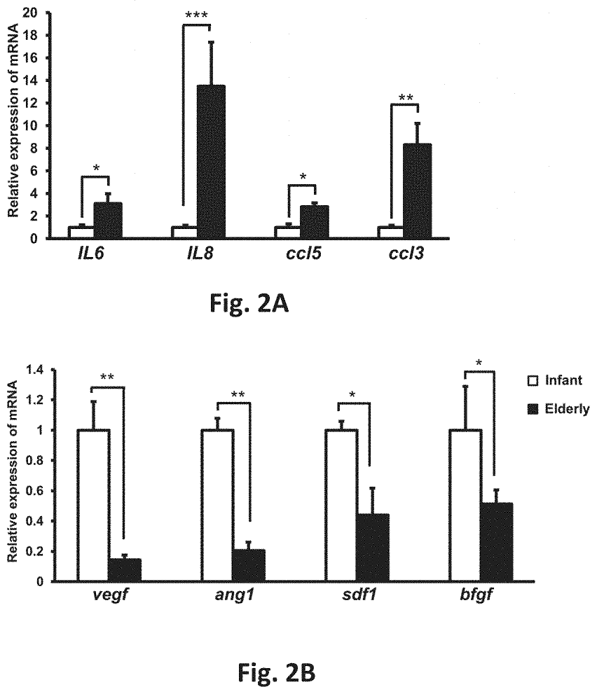

FIG. 2, panel 6C is a graphic depiction of the expression of ACE2 in Calu-3 human lung epithelial cells cultured in the presence of 0, 10 mM, 20 mM or 30 mM glucose for 24 hours.

FIG. 26, panel D is a graphic depiction of the expression of pro-inflammatory cytokines (IL-6, TNFα and IFN-β), in Calu-3 human lung epithelial cells cultured in the presence of 10 mM glucose for 0, 24, 48 or 72 hours.

FIG. 26, panel E is a graphic depiction of the expression of ACE2 in Calu-3 human lung epithelial cells cultured in the presence of 10 mM glucose for 24, 48 or 72 hours.

FIG. 26, panel F is a graphic depiction of the expression of CCL17 in Calu-3 human lung epithelial cells cultured in the presence of 10 mM glucose for 24 hours (No induction) and exposed to SARS COV2 spike protein peptides (+Prot_S) for an additional 24 hours.

FIG. 26, panel G is a graphic depiction of the expression of the pro-inflammatory cytokines (TNFα, IL6, IFN1β, IFNγ3, IP-10, and CXCL9) in Calu-3 human lung epithelial cells cultured in the presence of 10 mM glucose for 24 hours No induction) and exposed to SARS COV2 spike protein peptides (+Prot_S) for an additional 24 hours.

FIG. 27, panel A presents microscopic images of Calu-3 human lung epithelial cells cultured in the presence of 10 mM glucose for 24 hours alone (10 mM no induction), Calu-3 cells treated with 30 pmol Prot_S for 24 hours (10 mM+Prot_S), Calu-3 cells treated with 30 pmol Prot_S for 24 hours and nEVs for an additional 24 hours (10 mM+Prot_S+nEVs), and Calu-3 cells treated with 30 pmol Prot_S for 24 hours and nWJ-EVs for an additional 24 hours (10 mM+Prot_S+nWJ-EVs). nEVs and WJ-EVs were labeled by PKH-26-red.

FIG. 27, panel B is a graphic depiction of the expression of CCL17 in Calu-3 human lung epithelial cells cultured in the presence of 10 mM glucose for 24 hours (10 mM no induction), Calu-3 cells treated with 30 pmol Prot_S for 24 hours (10 mM+Prot_S), Calu-3 cells treated with 30 pmol Prot_S for 24 hours and nEVs for an additional 24 hours (10 mM+Prot_S+nEVs), and Calu-3 cells treated with 30 pmol Prot_S for 24 hours and nWJ-EVs for an additional 24 hours (10 mM+Prot_S+nWJ-EVs).

FIG. 27, panel C is a graphic depiction of the expression of the pro-inflammatory cytokines (TNFα, IL6, IFN1β, IFNγ3, IP-10, and CXCL9) in Calu-3 human lung epithelial cells cultured in the presence of 10 mM glucose for 24 hours (10 mM no induction), Calu-3 cells treated with 30 pmol Prot_S for 24 hours (10 mM+Prot_S), Calu-3 cells treated with 30 pmol Prot_S for 24 hours and nEVs for an additional 24 hours (10 mM+Prot_S+nEVs), and Calu-3 cells treated with 30 pmol Prot_S for 24 hours and nWJ-EVs for an additional 24 hours (10 mM+Prot_S+nWJ-EVs).

EVs evaluated in the results depicted in FIG. 27, panels A, B and C were isolated from four independent cell lines of MSCs, n=4. The data represent the mean±SD. ***P<0.01, **P<0.01, *P<0.05, ns: no significance. The experiments were performed in triplicate.

### DETAILED DESCRIPTION

All patents, publications, and patent applications cited in this specification are herein incorporated by reference as if each individual patent, publication, or patent application was specifically and individually indicated to be incorporated by reference in its entirety for all purposes.

Unless defined otherwise, all technical and scientific terms used herein have the same meaning as commonly understood by one of ordinary skill in the art to which the disclosure pertains. Other methods and materials similar, or equivalent, to those described herein can be used in the practice of the present disclosure.

In providing the present disclosure, the following terminology will be used in accordance with the definitions set out below.

As used in this specification and the appended claims, the use of the terms “a”, “an”, “the”, and similar referents in the context of describing the disclosed embodiments (especially in the context of the claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined, even if there is something intervening. The phrase “based on” should be understood to be open-ended, and not limiting in any way, and is intended to be interpreted or otherwise read as “based at least in part on,” where appropriate. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

### Definitions

The term “acute respiratory distress syndrome (ARDS) is a life-threatening inflammatory lung condition associated with significant morbidity and mortality. It occurs when fluid fills up the air sacs of the lungs which can lower the amount of oxygen or increase the amount of carbon dioxide in the bloodstream. ARDS can prevent organs from getting the oxygen they need to function, and can eventually cause organ failure. ARDS can develop as a complication of COVID-19, sepsis and pneumonia, and is a leading cause of death associated with the conditions.

The term “apoptotic bodies” is used herein with reference to a type of extracellular vesicle with a diameter of from 1000 to 5000 nm that are specifically released by cells undergoing apoptosis.

The term “effective amount” or “therapeutically effective amount” refers to the amount of a therapeutic agent that is sufficient to effect beneficial or desired results. The therapeutically effective amount may vary depending upon one or more of, the subject and condition being treated, the weight and age of the subject, the severity of the condition, the manner of administration and the like.

The terms “one embodiment”, “an embodiment” and “some embodiments”, mean that a particular feature, structure or characteristic described in connection with the embodiment may be included in at least one embodiment of the present disclosure. Thus, the use of the terms “in one embodiment”, “in an embodiment”, or “in some embodiments”, in various places throughout the specification do not necessarily refer to the same embodiment. Certain features of the disclosure, described in the context of separate embodiments, may also be provided in combination in a single embodiment. Conversely, features of the disclosure, which are, described in the context of a single embodiment, may also be provided separately or in one or more sub-combinations.

The term “exosome” is used herein with reference to an extracellular vesicle of heterogeneous multivesicular origin that are from 20 nm to 100 nm in diameter and contain mRNA, miRNA, DNA and proteins. Markers include TSG101 and CD63.

The term “extracellular vesicle” or “EV”, is used herein with reference to a heterogeneous membrane vesicle of endosomal and plasma membrane origin ranging from 30 to 5,000 nm in size. EVs includes exosomes, microvesicles, and apoptotic bodies. EVs are released under physiological conditions, upon cellular activation, senescence, and apoptosis.

As used herein, the term “heterogeneous” is used herein with reference to a population of cell derived EVs, e.g., exosomes or MVs, that differ in size and may have differing amounts of an exogenous nucleic acid, and/or differing amounts of an exogenous protein.

The term “homogeneous” is used herein with reference to a population of cell derived EVs, e.g., exosomes or MVs, that have a similar amount of an exogenous nucleic acid, a similar amount of an exogenous protein, and are of a similar size. For example, a homogenous population is one wherein at least about 90%, at least about 95%, at least about 98%, or 100% of the cell-derived exosomes or MVs are of a similar size.

The terms “proinflammatory cytokines” and “inflammatory cytokines” are used interchangeably herein with reference a type of signaling molecule (a cytokine) that promotes inflammation. They play an important role in mediating the innate immune response and are involved in the upregulation of inflammatory reactions.

The term “mesenchymal stem cells” or “MSCs” as used herein refers to a population of non-hematopoietic, multipotent stem cells that have the ability to differentiate into certain lineages, such as mesodermal lineages, endodermal lineages, and ectodermal lineages.

The term “microvesicle” or “MV” is used herein with reference to extracellular vesicles of plasma membrane origin that are 100 to 1000 nm in diameter and contain mRNA, miRNA, DNA, and proteins. Markers include TSG101 and CD63.

The term “MSC-derived EVs” is used herein with reference to extracellular vesicles derived from mesenchymal stem cells (MSCs). MSC-derived exosomes and MVs are subsets of MSC-derived EVs.

The term “purified population”, as used herein refers to cell populations, such as cell derived EVs, exosomes, MVs, or miRNA, that have undergone one or more processes of selection for the enrichment or isolation of the desired EVs, exosomes, MVs, or miRNA population relative to some or all of some other component with which EVs, exosomes, or MVs, are normally found. Alternatively, “purified” can refer to the removal or reduction of residual undesired components. Cells, EVs, exosomes, MVs, and miRNA described herein can be provided in isolated, purified, homogeneous, substantially homogeneous and/or heterogenous forms.

The term severe acute respiratory syndrome (SARS) is a viral respiratory illness caused by a coronavirus.

The terms “subject”, “individual”, and “patient” are used interchangeably herein to refer to a vertebrate, preferably a mammal, more preferably a human.

The term “substantially” refers to the complete or nearly complete extent or degree of a characteristic and in some respects, defines the purity of the isolated or purified population of cell derived EVs, exosomes, or MVs.

The terms “therapeutic agent” and “cellular therapy agent” are used herein with reference to EVs, specifically exosomes and MVs, molecules, and compounds that confer some beneficial effect upon administration to a subject. The beneficial effect may be diagnostic determinations; treatment of a disease, symptom, disorder, or condition; reducing or preventing the onset of a disease, symptom, disorder or condition; and generally counteracting a disease, symptom, disorder or condition.

The terms “treatment”, “treating”, “palliating”, and “ameliorating” may be used interchangeably herein with reference to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “Wharton's Jelly”, as used herein refers to a mucous-connective tissue substance found in the umbilical cord. The components of Wharton's Jelly include mucous connective tissue, which comprises myofibroblasts, fibroblasts, macrophages, mesenchymal stem cells and an amorphous substance composed of hyaluronic acid and possibly other yet uncharacterized cell populations. Wharton's Jelly is a component of the umbilical cord.

The term “wound healing” is used herein with reference to the body's process of repairing damaged tissue. There are many situations, when tissue repair fails, leading to chronic non-healing wounds, for example, in the case of diabetic ulcers and bedsores.

### EMBODIMENTS

In some embodiments, the disclosure provides EV compositions derived from stem cells, for example, mesenchymal stem cells (MSCs), embryonic stem cells, or pluripotent stem cells.

In some embodiments, the EV composition is obtained by enrichment and culture of MSCs.

In some embodiments, the MSCs are derived from adipose tissue.

In some embodiments, the adipose tissue is adult adipose tissue.

In some embodiments, the adipose tissue is infant adipose tissue.

In some embodiments, the MSCs are adult MSCs.

In some embodiments, MSCs are obtained from dental pulp, placenta, umbilical cord tissue, or amniotic membrane tissue.

In some embodiments, the MSCs are fetal/perinatal MSCs.

In some embodiments, MSCs are obtained from Wharton's Jelly.

The EV composition may be characterized using one or more techniques including electron microscopy, atomic force microscopy (AFM), dynamic light scattering (DLS), nanoparticle tracking analysis (NTA), tunable resistive pulse sensing (TRPS), flow cytometry, analysis of protein content, e.g., by Western blot, analysis of nucleic acid content, for example, RNA content such as micro RNA content.

In some embodiments, the EV composition is characterized for the relative percentage of exosomes and microvesicles.

In some embodiments, the EV composition is separated into exosome and microvesicle fractions.

In some embodiments, the exosomes have a diameter of from about 20 nm to 100 nm.

In some embodiments, the exosomes and/or MVs express TSG101 and CD63 proteins.

In some embodiments, the MVs have a diameter of from about 100 nm to 1000 nm.

In some embodiments, the EV composition comprises exosomes and MVs combined in a ratio that differs from the ratio of exosomes to MVs found in cells, for example, MSCs.

In some embodiments, the EV composition comprises exosomes and MVs combined in a ratio of from about 0.8:1 to about 1:1.3, from about 1:1.3 to about 0.8:1, from about 1:1 to about 1.5, from about 1:1 to about 2:1, or from about 1:0.5 to about 1.2.5, e.g., about 1:1, 1:1.5, 1:2, 1:2.5, 1:3, 1:4, 1:5, 1:6, 1:7, 1:8, 1:9, 1:10, 10:1, 9:1, 8:1, 7:1, 6:1, 5:1, 4:1, 3:1, 2:1, or 1.5:1.

In some embodiments, the disclosure provides EV compositions and methods of use thereof in treating skin disorders and skin wounds.

In some embodiments, the EV composition is used to treat diabetic ulcers, pressure ulcers, e.g., bed sores, burns, skin diseases, or skin cancers.

MSCs from patients with Type 2 diabetes (T2DM MSCs) are known to have impaired function, e.g., impaired wound healing ability. In such cases, the wound healing ability may be less than 80% the wound healing ability of normal MSCs, less than 70%, less than 60%, less than 50%, less than 40%, less than 30%, or about 20%, about 30%, about 40%, about 50%, about 60%, or about 70% of the wound healing ability of normal MSCs.

In some embodiments, wound healing of skin cells from patients with Type 2 diabetes is improved by at least about 10%, about 20%, about 30%, about 40%, about 50%, about 60%, about 70%, or about 80% or more by treating the skin wound with an EV composition comprising exosomes and MVs. Wound healing ability is determine by size of the necrotic area in a murine model.

In some embodiments, the wound healing of skin cells from patients with Type 2 diabetes is restored to that of skin cells from patients without Type 2 diabetes by treating the skin wound with an EV composition comprising a mixture of exosomes and MVs.

In some embodiments, the time for wound healing of skin cells from patients with Type 2 diabetes is decreased by about 20%, about 30%, about 40%, about 50%, about 60%, about 70%, about 80%, about 90%, about 100% or more by treating the skin wound with an EV composition comprising a mixture of exosomes and MVs.

In some embodiments, the size of a necrotic lesion caused by a skin disorder or skin wound is decreased by at least 10%, at least 20%, at least 30%, at least 40%, at least 50%, at least 60%, at least 70%, at least 80% or more within 5 to 14 days, or about 5 days, about 6 days, about 7 days, about 8 days, about 9 days, about 10 days, about 11 days, or within about 14 days following treatment of the skin wound with an EV composition comprising a mixture of exosomes and MVs.

In some embodiments, a composition comprising exosomes and MVs in a ratio of from about 0.5:1.5 or about 1:1 is more effective than the wound healing ability of a composition comprising exosomes and MVs in a ratio of 1:2 or 2:1, as determined by μg of protein in the sample.

Edaravone is a free radical scavenger that eliminates lipid peroxides and hydroxyl radicals and is FDA-approved for treatment of Amyotrophic Lateral Sclerosis (ALS).

In some embodiments, the wound healing ability wound healing of skin cells from patients with Type 2 diabetes is improved by at least about 10%, about 20%, about 30%, about 40%, about 50%, about 60%, about 70%, or about 80% or more by treating the skin wound with an EV composition derived from MSCs treated with Edaravone. Wound healing ability is determine by survival time in a murine model.

In some embodiments, an EV composition stored at −30° C. for at least 6 months or at −80° C. for at least 1 year exhibits no change in wound healing ability.

In some embodiments, an EV composition, such as a combination of exosomes and MVs is administered by injection, for example, injection at the wound site, or intravenous injection.

In some embodiments, an EV composition, such as a combination of exosomes and MVs is administered by direct application, for example, applying the EV composition to the wound site by “dripping”.

The disclosure contemplates use of EVs immediately after isolation, or alternatively short- and/or long-term storage of EVs, for example, in a cryopreserved state prior to use. Proteinase inhibitors are typically included in freezing media as they provide exosome integrity during long-term storage. Freezing at −20° C. is not preferable since it is associated with increased loss of exosome activity.

The disclosure includes methods and compositions for treating a patient, by administering at least one therapeutically effective dose of EVs to a patient, wherein the patient is afflicted with a lung condition such as COVID-19, over-reactive inflammatory responses, cytokine storms and/or ARDS.

In some embodiments, the EV composition is used to treat overactivation of the immune system, e.g., a cytokine storm. Overexpression of cytokines or a “cytokine storm” may be induced by viral infection and can result in ARDS.

In some embodiments, the cytokine storm is characterized by expression of cytokines including but not limited to TNFα, IL6, IFN1β, IFNγ3, IP-10 (also referred to as CXCL10 (interferon-γ inducible protein of 10 kDa), and CXCL9.

In some embodiments, the EV composition is provided as prophylactic treatment for a subject likely to experience lung conditions such as COVID-19, an over-reactive inflammatory response, a cytokine storm and/or ARDS.

In some embodiments, the EV composition is provided as early treatment for a subject experiencing early stages of a lung condition such as COVID-19, an over-reactive inflammatory response, a cytokine storm and/or ARDS.

The mesenchymal stem cells, and thus the EVs, contemplated for use in these methods may be derived from the same subject to be treated (autologous) or they may be derived from a different subject preferably of the same species (allogeneic).

The EVs may be administered by any route that effects delivery to the lungs. Systemic administration routes such as intravenous bolus injection or continuous infusion are suitable. More direct routes such as intranasal administration, intratracheal administration (e.g., via intubation), and inhalation (e.g., via an aerosol through the mouth or nose) are also contemplated and in some instances may be more appropriate particularly where rapid action is necessary.

The EVs may be provided as a pharmaceutical composition.

In some embodiments, the EVs are administered by aspirating the pharmaceutical composition into at least one lung of the patient.

Pharmaceutical compositions for injection may be presented in unit dosage form, e.g., in ampoules or in multi-dose containers, with or without an added preservative.

The pharmaceutical compositions may take such forms as water-soluble suspensions, solutions or emulsions in oily or aqueous vehicles, and may contain agents such as suspending, stabilizing and/or dispersing agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension. Optionally, the suspension may also contain suitable stabilizers or agents which increase solubility. Alternatively, the EVs may be in lyophilized or other powder or solid form for constitution with a suitable vehicle before use.

Pharmaceutical compositions may be administered according to any desired dosage regimen, such as once per day, once every 2 days, once every 3 days, once every 4 days, once every 5 days, once every 6 days, once every 7 days, once every 8 days, once every 9 days, once every 10 days, once every 11 days, once every 12 days, once every 13 days, once every 14 days, more than once per day, or even multiple times per day, depending upon, among other things, the condition of the patient being treated and the judgment of the prescribing physician.

In some embodiments, the EV composition is administered 2 times, 3 times, 4 times, 5 times, 6 times, 7 times, 8 times, 9 times, or 10 or more times.

In some embodiments, the EV composition is administered on one or more of days 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

The EVs may be administered with one or more secondary therapeutic agents. As used herein, a therapeutic agent refers to any agent which can be used in the prevention, treatment and/or management of a lung disease such as those discussed herein. It is to be understood that such secondary therapeutic agents may be administered by any suitable route including oral administration, intranasal administration, intratracheal administration, inhalation, intravenous administration, etc. Those of ordinary skill in the art will know the customary routes of administration for such secondary agents.

In some embodiments, the EV compositions are effective to reduce SARS COV2 spike protein peptide-induced pro-inflammatory cytokine overexpression in lung cells.

In some embodiments, the EV compositions are effective to reduce SARS COV2 spike protein peptide-induced pro-inflammatory cytokine overexpression in lung cells that was further increased by exposure to high levels of glucose.

In some embodiments, the WJ EV compositions are effective to increase CCL17 expression in lung cells exposed to SARS COV2 spike protein peptides.

In some embodiments, the WJ EV compositions are effective to increase CCL17 expression in lung cells exposed to SARS COV2 spike protein peptides that was further decreased by exposure to high levels of glucose.

In some embodiments, EV compositions including exosomes and MVs in a ratio of about 1:1.5 exosomes to MVs are effective to reduce SARS COV2 spike protein peptide-induced pro-inflammatory cytokine overexpression.

In some embodiments, EV compositions including exosomes and MVs in a ratio of about 1:1.5 exosomes to MVs are effective to increase CCL17 expression in lung cells exposed to SARS COV2 spike protein peptides.

### Utility.

Treatment of Skin disorders and Skin Wounds. Acute and chronic skin injuries (wounds), resulting from burns, pressure, Diabetes mellitus, and venous stasis, pose a great burden on society. Normal wound healing is a complicated biological process that requires the accurate cooperation of many types of cells and coordination of biological and molecular events.

The difficulty in healing cutaneous wounds and ulcers is a significant health problem that impacts the quality of life for millions of people. It has been suggested that stem cells and progenitor cells can promote wound healing and that secreted factors associated with EVs contribute to observed therapeutic benefits. Local and systemic administration of EVs have been shown to promote wound healing in animal models of cutaneous wounds. In addition, experimental evidence suggests that EV-mediated transfer of proteins and RNA triggers pro-repair pathways in target cells.

Diabetes mellitus is a metabolic disease that affects more than 340 million individuals worldwide and about 20% of them develop diabetic wounds (often referred to as “ulcers”). Leg and foot ulcers are the most common wounds affecting diabetic patients. The incidence of delayed healing in diabetic patient is increasing due to lack of preventive and control measures. (Patel, Satish, Biomedicine and Pharmacotherapy, Volume 112, April 2019). Diabetic foot ulcers are a severe complication of diabetes, preceding most diabetes-related amputations. Diabetic foot ulcers require over billions of dollars yearly for treatment and are now a global public health issue. Recently, stem cell therapy has emerged as a new interventional strategy to treat diabetic foot ulcers and appears to be safe and effective in both preclinical and clinical trials (Lopes L, et al., Stem Cell Res Ther. 9(1):188, 2018). The compositions and methods described herein find utility in treatment of diabetic leg and foot ulcers.

Pressure ulcers. Pressure ulcers are a type of skin injury (wound) where the skin and underlying tissue is placed under constant pressure for enough time to cause tissue ischemia, loss of nutrition and oxygen supply to the tissue, and eventually tissue necrosis. Pressure ulcers, such as bed sores are very difficult to treat. The compositions and methods described herein find utility in treatment of pressure ulcers such as bed sores.

Burns. Sunburn and small burns can often be treated at home, however, extensive burns, and chemical or electrical burns need intensive medical treatment. EVs obtained from cells, such as MSCs, have been evaluated and shown efficacy in different animal models of skin injury, including wound healing models in healthy and diabetic mice and severe burn models in rats (Carrasco, Elisa et al. Int. journal of Mol. Sciences, Vol. 20,11 2758, Jun. 5, 2019). This suggests that an EV composition containing both exosomes and MVs has potential for treatment of burns. The compositions and methods described herein find utility in treatment of burns.

Skin diseases. Common skin diseases include acne, eczema, seborrheic dermatitis, and psoriasis. A variety of treatments are available for these conditions and their use meets with a range of success. Studies showing the efficacy of EVs on treatment of skin diseases, for example EVs obtained from MSCs, suggest that an EV composition containing both exosomes and MVs has potential for treatment of a variety of skin diseases. The compositions and methods described herein find utility in treatment of skin diseases.

Skin cancers. There are three major types of skin cancers: basal cell carcinoma (BCC), squamous cell carcinoma (SCC), and melanoma. Treatment for non-melanoma skin cancer typically includes surgical removal of the cancer cells, while treatment for melanoma may include surgery, radiation therapy, chemotherapy, and immunotherapy. These treatments have met with variable efficacy. EVs carry a large array of bioactive EVs have been reported to have unique molecular signatures in cancer patients, and are the subject of numerous research programs directed to diagnosis and prognosis for various types of cancer (Han, L, et al. Mol. Cancer 18, 59, 2019). Early studies have shown that cancer cells secrete more EVs than their non-malignant counterparts, and EVs can be isolated from body fluids, suggesting the potential of EVs as biomarkers for diagnosis, and monitoring of cancer (Xu, R., et al. Nat Rev Clin Oncol 15, 617-638, 2018). In one study, the ability of EVs secreted by melanoma cells in response to chemotherapy modulate tumor response to alkylating drugs was evaluated, and the results showed that in vitro human and murine melanoma cells secrete more EVs after treatment with temozolomide and cisplatin, suggesting that EVs play a role in treatment failure in melanoma cells (Andrade, L. N. d. S., et al., Sci Rep 9, 14482, 2019). These studies and many others suggest a role for EV compositions in the diagnosis and prognosis of skin cancers and that an EV composition containing both exosomes and MVs, has the potential to improve currently available treatments for skin cancers. The compositions and methods described herein find utility in treatment of skin cancer.

Pneumonia, ARDS, Cytokine Storms, and COVID-19. Since the pandemic in 2019, SARS-CoV-2 has infected tens of millions of people worldwide. One factor contributing to organ failure and the death of patients is over expression of cytokines or a “cytokine storm” induced by viral infection and resulting in ARDS, also known as respiratory distress syndrome (RDS).

Patients with diabetes mellitus experience more severe symptoms and higher mortality after SARS-CoV-2 infection.

The administration of MVs and exosomes to modulate COVID-19, over-reactive inflammatory responses, cytokine storms and/or ARDS finds utility in the treatment of any subject likely to derive benefit therefrom, including human subjects, dogs, cats, cows, pigs, horses and the like.

The administration of MVs and exosomes to modulate COVID-19, over-reactive inflammatory responses, cytokine storms and/or ARDS finds further utility in in the treatment of any subject that has a high glucose level, such as a subject with diabetes.

Intravenous infusion of MSCs has been shown to improve the outcome for 7 patients with COVID-19 pneumonia. It has been proposed that MSC treatment can inhibit the overactivation of the immune system, improve the pulmonary microenvironment, protect alveolar epithelial cells, prevent pulmonary fibrosis and improve lung function (Leng Z., et al., Aging and disease, 2020, Vol. 11 (2): 216-228).

EVs have been reported to play a role in inflammatory lung injury in ARDS (Mahida, R Y, et al. Am J Respir Cell Mol Biol. 2020 Feb 28). Unfortunately, the current treatment for ARDS is mainly supportive. MSCs are being explored and have exhibited positive results in animal studies. It has been proposed that the therapeutic effects of MSCs may be partially due to release of EVs (Shah, T G, et al. Clin Transl Med. 2019; 8: 25).

Symptoms of ARDS include one or more of labored and rapid breathing, muscle fatigue and general weakness, low blood pressure, discolored skin or nails, a dry, hacking cough, a fever, headaches, a fast pulse rate and mental confusion. A cytokine storm is characterized by overactivation of the immune system. Symptoms of COVID-19 include a cough, shortness of breath or difficulty breathing, at least two of fever, chills, repeated shaking with chills, muscle pain, headache, sore throat, and new loss of taste or smell. more severe symptoms include trouble breathing, persistent pain or pressure in the chest, new confusion or inability to arouse, and bluish lips or face.

The compositions and methods described herein find utility in the treatment and reduction of the symptoms associated with pneumonia, ARDS, cytokine storms, and COVID-19.

### Examples

The disclosure is further illustrated by the following examples. The examples are provided for illustrative purposes only. They are not to be construed as limiting the scope or content of the disclosure in any way.

### Example 1. Enrichment or Isolation of MSCs

A. Enrichment or Isolation of adipose tissue-derived mesenchymal stem cells (AT-MSCs). Enrichment or isolation of adipose tissue-derived mesenchymal stem cells (AT-MSCs) was carried out using human adipose tissues obtained from the Department of Cardiovascular Surgery, University of Tsukuba Hospital, including infants (1-12 months old) and elderly donors (70-80 years old). AT-MSCs were isolated from adipose tissues according to a previously described method (Nagano M et al, Stem Cells Dev 19:1195-1210, 2010). Briefly, adipose tissues were digested with 0.1% collagenase (Invitrogen) in PBS then centrifuged to harvest the cells, and re-suspended in culture medium, Iscove's Modified Dulbecco Medium (IMDM, Invitrogen), supplemented with 10% fetal bovine serum (FBS, Invitrogen), 2 mg/ml L-glutamine (Invitrogen), 100 units/ml penicillin (Invitrogen) and 5 ng/ml bFGF (Peprotech, UK). All AT-MSCs used were from passage 3 to 8.

B. Enrichment of Wharton Jelly-Derived Mesenchymal Stem Cells (WJ-MSCs).

Enrichment or isolation of WJ-MSCs was carried out using human umbilical cord samples obtained from the Department of Obstetrics and Gynecology, University of Tsukuba Hospital. Briefly, the umbilical cords were washed with PBS to remove blood, cut into 5 cm-long pieces. The amnion was cut along the horizontal axis and the inside blood vessels were removed from the surrounding tissues. Then, the umbilical cord tissues were collected and cut into small pieces following by the digestion with 0.1% collagenase (Invitrogen) in PBS for 30 minutes, then centrifuged to harvest the cells, and re-suspended in culture medium, Iscove's Modified Dulbecco Medium (IMDM, Invitrogen), supplemented with 10% fetal bovine serum (FBS, Invitrogen), 2 mg/ml L-glutamine (Invitrogen), 100 units/ml penicillin (Invitrogen) and 5 ng/ml bFGF (Peprotech, UK). The medium was replaced every three days and the WJ-MSCs attached to the plate were grown up to 80% confluence before doing serial passages.

### Example 2. Reactive Oxygen Species (ROS) in Elderly AT-MSCs Versus Infant AT-MSCs and Effect of Antioxidant Treatment

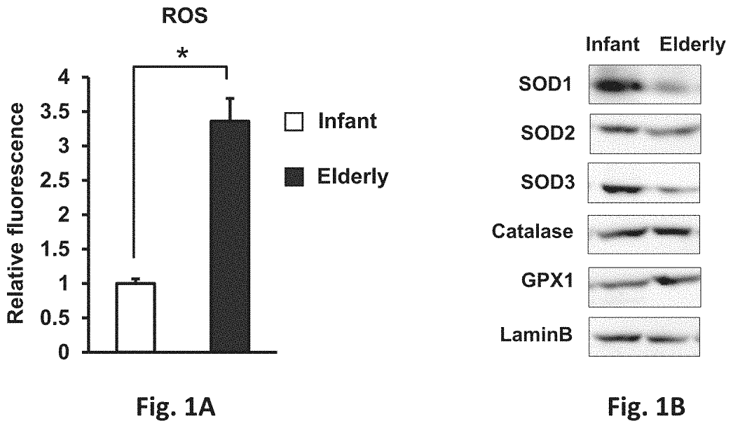

The expression of ROS in infant- and elderly-derived ATMSCs was evaluated. The intracellular ROS level was measured using 2′,7′-dichlorodihydrofluorescein diacetate (H2-DCFDA, Invitrogen) according to the manufacturer's instructions. Briefly, after reaching 80% confluence, the ATMSCs were washed with PBS and incubated with PBS containing 10 μM H2-DCFDA at 37° C. for 30 minutes. The fluorescence intensity was analyzed by absorbance at 495 nm and 525 nm wavelengths using a photoemission spectrophotometer (Corona Electric, Japan). The results showed a 3.36-fold higher ROS level in elderly-derived AT-MSCs, compared to infant-derived ATMSCs (FIG. 1A). The expression of antioxidant genes was also evaluated, and the results showed that antioxidant gene products including SOD1 and SOD3 proteins were lower in the elderly derived ATMSCs. SOD1 exhibited a 2.3-fold lower protein level in elderly-derived ATMSCs, and SOD3 exhibited a 2.5-fold lower protein level (n=4, p<0.05) as compared to infant-derived ATMSCs (FIG. 1B).

The effects of aging and accumulation of ROS on the functions of AT-MSCs, were evaluated by analyzing the mRNA expression of growth factors in infant and elderly AT-MSCs. The results showed increased expression of the proinflammatory cytokines (IL6 and IL8), and chemokines (CCL5 and CCL3) in elderly AT-MSCs compared to infant AT-MSCs (FIG. 2A). In contrast, other homeostatic factors, such as angiogenic factors (VEGF, ANG1, and bFGF) and stromal cell-derived factor 1 (SDF1), exhibited a lower level of expression in elderly AT-MSCs compared to infant AT-MSCs (FIG. 2B). In FIGS. 2A and 2B, *=P<0.05, **=P<0.01, and ***=P<0.001.

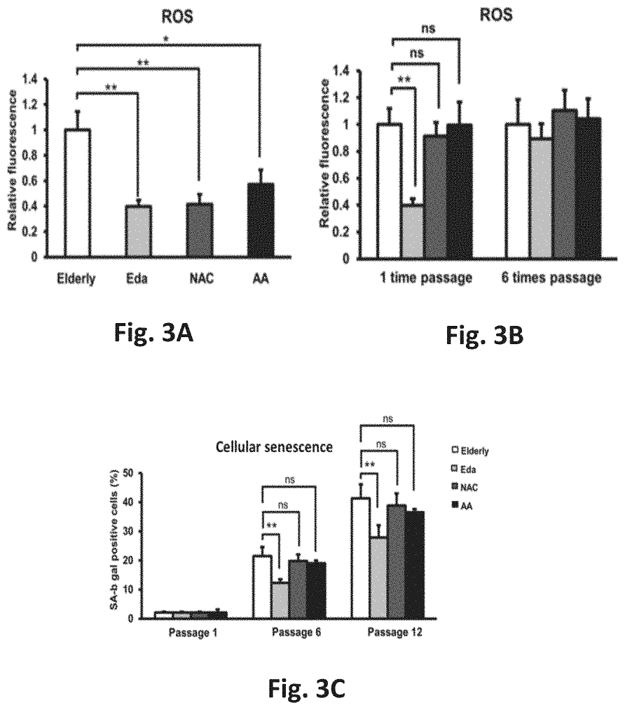

While not wishing to be bound by theory, the oxidative stress and resulting accumulation of ROS are believed to relate to the impaired function of elderly AT-MSCs. The effects of anti-oxidative agents on elderly AT-MSCs were tested and the expression of ROS in elderly AT-MSCs were measured following culture in the presence of 20 μM Edaravone (Eda), 1 mM N-acetylcysteine (NAC), or 2 μM Ascorbic Acid (AA), respectively. While all 3 agents reduced ROS levels after 24 hours of culture (FIG. 3A), the ROS reducing effects of Eda were evident following 1 cell passage (FIG. 3B). The concentration of Eda, NAC and AA was maintained in the culture media throughout the culture period. The effects of anti-oxidative agents on cell senescence were also investigated. Cell senescence was assessed by measuring the activity of senescence associated (SA) β-galactosidase following 6 or 12 passages of culture in the presence of 20 μM Eda, 1 mM NAC, or 2 μM AA. Only cells treated with Eda exhibited reduced (SA) β-galactosidase activity, indicating it lowered cell senescence in elderly AT-MSCs (FIG. 3C). In FIGS. 3A, 3B and 3C, *=P<0.05, **=P<0.01, and ns=not significant.

The effect of the anti-oxidative agents Eda, NAC and AA on the wound healing ability of elderly AT-MSCs was investigated using the mouse punch biopsy model, as described in Dunn et al., J Vis Exp. (75): 50265, 2013). Briefly, 7-week-old male C57BL/6 mice were anesthetized using avertin and shaved. Two wounds were established using 5 mm biopsy punch in the mice upper dorsal area. MSCs (5×10⁵ cells/wound) or EVs (4 μg/wound) were injected at 4 points surrounding the punch area. Each mouse had two punched wounds, (1) PBS and (2) cells or EV-treatment. A splint was stitched to the surrounding wound areas and covered with Tegaderm (3M, USA), and the wound sites were observed every day. The images of the wound areas were captured, and the necrotic areas were analyzed using Image J software (NIH, MD).

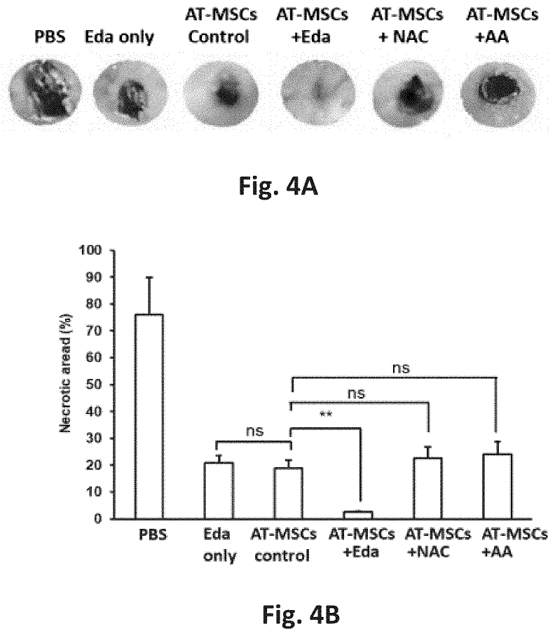

Elderly AT-MSCs were cultured for 24 hours in the presence of 20 μM Edaravone (Eda), 1 mM N-acetylcysteine (NAC), or 2 μM Ascorbic Acid (AA), then the effect on wound healing was evaluated. As shown in FIGS. 4A and 4B, treatment of AT-MSCs with 20 μM Eda significantly improved wound healing while NAC and AA had no effect. In FIG. 4B, **=P<0.01, and ns=not significant.

### Example 3. Wound Healing Ability of Elderly AT-MSC Versus Infant AT-MSCs

It was hypothesized that the impaired ischemic wound healing ability of elderly AT-MSCs and their derived extracellular vesicles (EVs) was related to impaired vascularization. The effects of aging on the neovascularization functions of ATMSCs was evaluated using the ischemic skin flap mouse model.

Streptozocin-induced type 1 diabetic mice were used for this study. Briefly, The 10-week-old C57BL/6 mice were subjected to intraperitoneal injection of streptozotocin (40 mg/kg) once daily for 5 days. A week after injection, the blood glucose was confirmed to be a level higher than 11.1 mmol/L. The mice then were anesthetized and the skin on the dorsal surface was cut into a peninsular-shaped incision (3 cm×2 cm) to create an ischemic gradient by blood flow restriction. The mice were divided into 3 groups: sham injection with PBS, infant AT-MSCs, and elderly AT-MSCs. On the first day after the surgical process, MSCs were injected locally with 5×10⁵ MSCs for each treatment group, respectively, at each of 4 locations of the flap skin. Cyclosporin-A (20 mg/kg body weight) was intraperitoneally injected into the mice every 2 days to induce immunosuppression. The healing effects were evaluated by immunostaining 3 days after injection and by necrotic area quantification 7 days after injection.

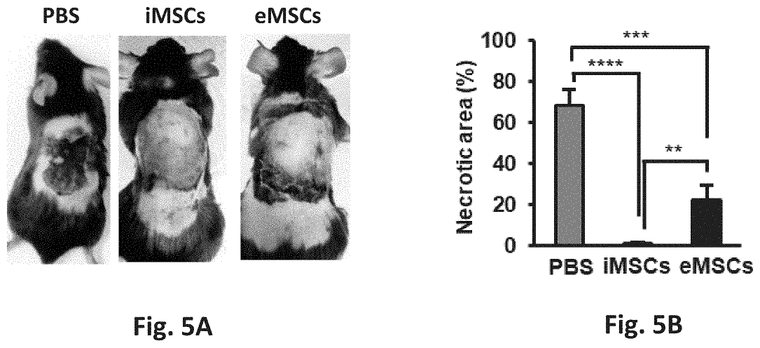

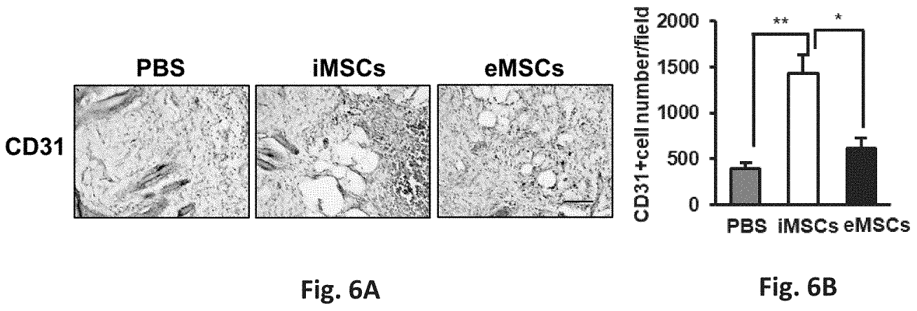

Seven days following injection, the wound area of infant AT-MSC (iMSC)-injected mice was almost completely healed as compared to the PBS-injected mice. In contrast, the necrotic area of elderly AT-MSC-injected mice was significantly greater than that of infant AT-MSC-injected mice (Necrotic area, PBS: 72.1%, iMSCs: 1.03%, eMSCs: 21.9%, FIGS. 5A and 5B). In FIG. 5B, **=P<0.01, ***=P<0.001, and ****=P<0.0001. In addition, immunohistochemical analysis for CD-31 expression indicated a greater number of CD31-positive vascular endothelial cells in iMSC-injected mice relative to eMSC-injected mice. These results suggest that eMSCs are less effective at promoting vascularization than iMSCs (FIGS. 6A and 6B). In FIG. 6B, *=P<0.05 and **=P<0.01.

### Example 4. Collection and Characterization of Extracellular Vesicles

Collection of AT-MSC-derived extracellular vesicles was carried out by seeding AT-MSCs at 10⁶ cells/plate and culturing for 12 hours. The culture medium was replaced with fresh IMDM containing 0.25% FBS and continued culturing for an additional 48 hours. The AT-MSC-CM (conditioned media) was collected by centrifuging at 1000 pm for 5 minutes, followed by centrifugation at 3000 rpm for 10 minutes at 4° C. to remove the cell debris. For AT-MSC-EV isolation, the AT-MSC-CM was ultracentrifuged at 37,000 rpm for 70 minutes at 4° C. The pellet was then resuspended in PBS and the protein concentration was measured using the Bradford assay.

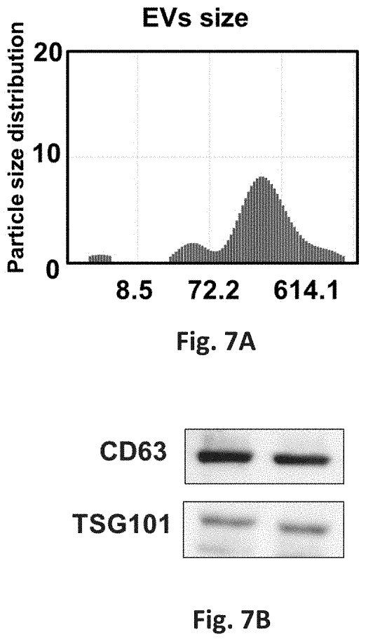

Extracellular vesicles were characterized using a Particle size Analyzer FDLS3000 (FIG. 7A), and by staining with anti-CD63 and anti-TSG101 antibodies and examination of the results by Western Blot (FIG. 7B).

### Example 5. Rejuvenation Elderly MSCs

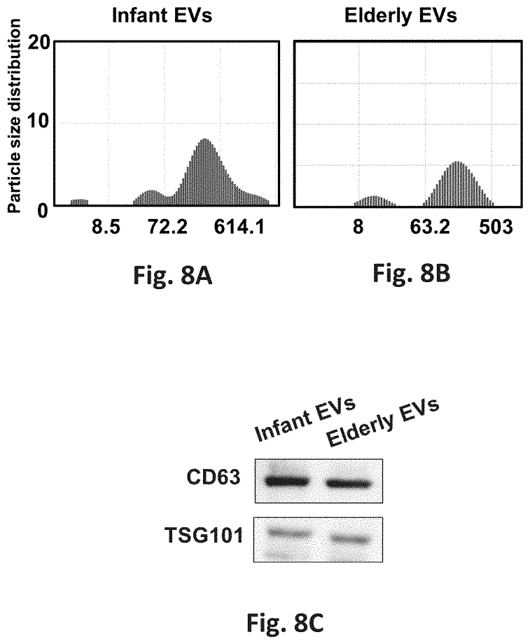

iEVs and eEVs were characterized in order to examine the effect of aging. The results showed that both iEV (FIG. 8A) and eEV (FIG. 8B) fractions contain both exosomes and MVs, as indicated by the particle size of from 10 to 700 nm, and that both fractions express CD63 and TSG101 (FIG. 8C). The relative percentage of exosomes and MVs in an iEV and eEV composition has been shown to vary depending upon the experimental conditions and source of the EVs.

Collectively, these data suggest that both iEVs and eEVs exhibit characteristics typical of EVs in terms of size and exosome marker expression.

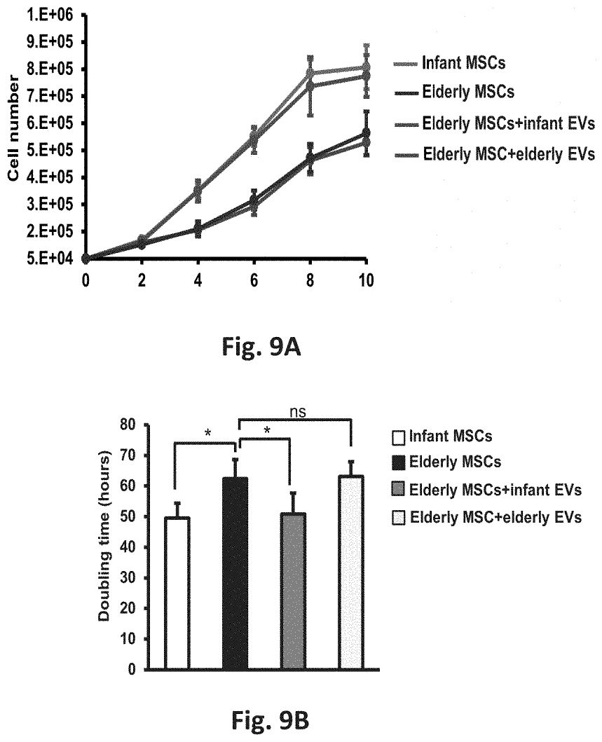

A study was performed in order to determine whether infant MSC extracellular vesicles (iMSC-EVs or iEVs) can rejuvenate and improve the functions of elderly MSCs (eMSCs). The effects of iEVs and eEVs on eMSCs were tested by incorporating either iEVs or eEVs into eMSCs by culturing them together. After confirming that 100% of target eMSCs in the population contained either iEVs or eEVs by the PKH26 signal under the microscope and by FACS analysis, the characteristics of EV-incorporated eMSCs were examined. The results showed that eEVs did not promote the proliferation of target eMSCs, whereas iEVs significantly increased the proliferation of target eMSCs to a level as high as iMSCs (FIG. 9A). For example, the doubling time in an hour was: iMSCs: 49.5, eMSCs: 62.4, iEV-incorporated eMSCs: 50.8, eEV-incorporated eMSCs: 63.1 (FIG. 9B). In FIG. 9B, *=P<0.05, and ns=not significant.

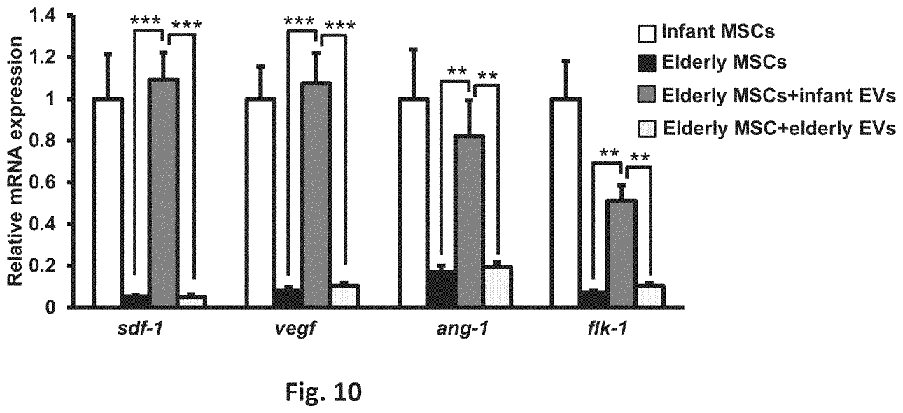

eMSCs with incorporated iEVs exhibited upregulated expression of angiogenic cytokines, while eMSCs with incorporated eEVs did not. A comparison of the level of angiogenic cytokine mRNA expression between eMSCs treated with iEVs and eEVs showed that the expression of sdf-1, vegf, ang-1, and flk-1 was significantly higher in those treated with iEVs compared to eEVs (FIG. 10). These data show the relative ability of iEVs and eEVs to modify the function of eMSCs. In FIG. 10, **=P<0.01, ***=P<0.001.

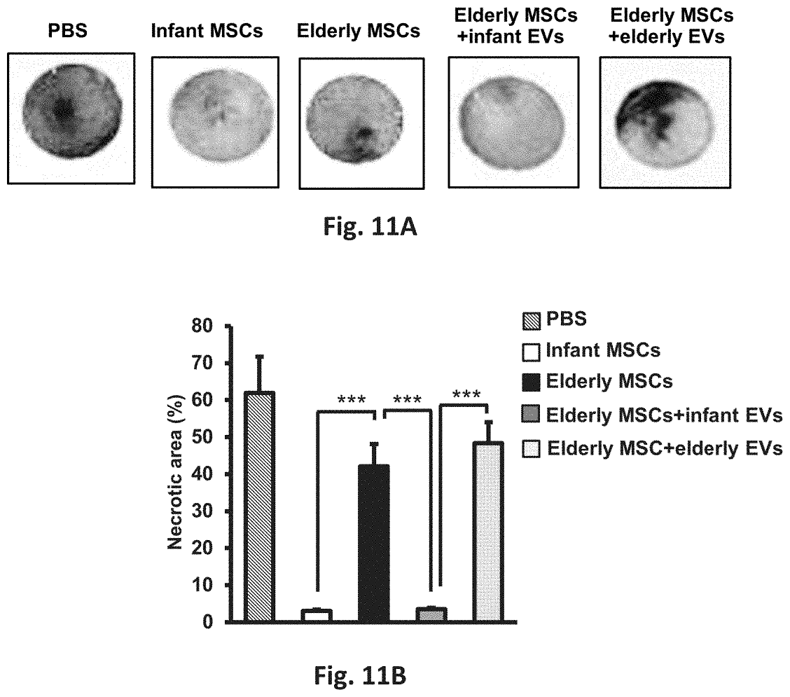

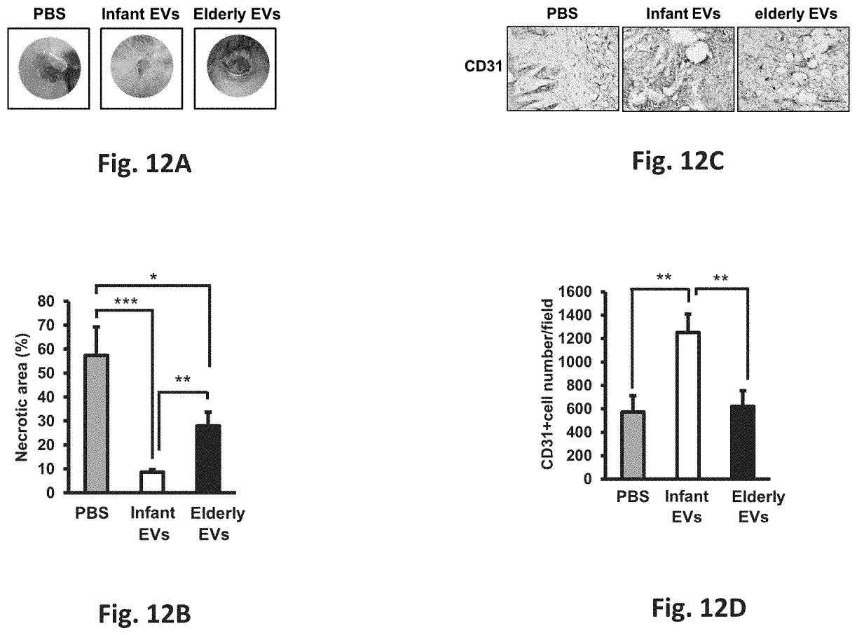

In addition, studies in the in vivo punch wound healing mouse model showed that adding iEVs to eMSCs significantly increased the ability of eMSCs to promote the wound healing process in punch mice, similar to the level of wound healing observed for iMSCs. In contrast, the poor wound healing ability of eMSCs was not improved by eEVs. The necrotic area of mice transplanted with iMSCs: 3.04%, eMSCs: 42.13%, eMSCs incorporated with iEVs: 3.53%, eMSCs incorporated with eEVs: 48.34%, n=6, p<0.001 (FIGS. 11A and 11B). In FIG. 11B, ***=P<0.001. The ability of MSC-EVs to form new blood vessels has been suggested. The in vivo functions of iEVs and eEVs were compared using the ischemic punch wound healing model in diabetic mice. Ten days following injection, EVs significantly promoted the wound healing process as demonstrated by a reduction in the necrotic area for the iEV treatment group to 8.6%, of the PBS control while the eEV treatment group exhibited only a 27.3% reduction in the necrotic area relative to control mice injected with PBS. Of note, although injection of eEVs showed the enhanced wound healing relative to treatment with PBS alone, the effects were significantly less than that of iEVs (FIGS. 12A and 12B). The neovascularization ability of iEVs and eEVs was compared by staining the skin tissues with CD31. Mice transplanted with iEVs showed significantly higher new vessel formation, determined by the high number of CD31-positive cells, compared to those injected with eEVs (FIGS. 12C and 12D). In FIGS. 12B and 12D, *=P<0.05, **=P<0.01, ***=P<0.001.

Taken together, these data suggest that iEVs can rejuvenate eMSCs by inducing proliferation and upregulating impaired cytokines, thereby promoting the healing functions of the eMSCs.

### Example 6. Rescue of Type 2 Diabetes AT-MSCs (T2DM MSCs) by Extracellular Vesicles

T2DM MSCs exhibit poor wound healing and increased expression of EGR1 and IL-6 mRNA as compared to normal MSCs. Experiments showed that EVs derived from healthy donor AT-MSCs were able to rescue the impaired wound healing ability of T2DM MSCs. The ability of T2DM MSCs treated with EVs derived from normal AT-MSCs was evaluated using an in vitro wound healing assay. An in vitro scratch assay was performed as follows: MSCs were seeded at a density of 3×10⁵ cells per 4-well plates. After 24 hours, confluent monolayers were treated with mitomycin C (10 mg/mL) for 3 hours to eliminate the possibility of cell proliferation. A scratch was created with a p1000 pipet tip (1 mm width) and cells were untreated or treated with EVs in the culture medium. Images of the scratch were captured at 0 h and 16 hours. The analysis of the scratch area was performed using the WimScratch software program (https://mywim.wimasis.com).

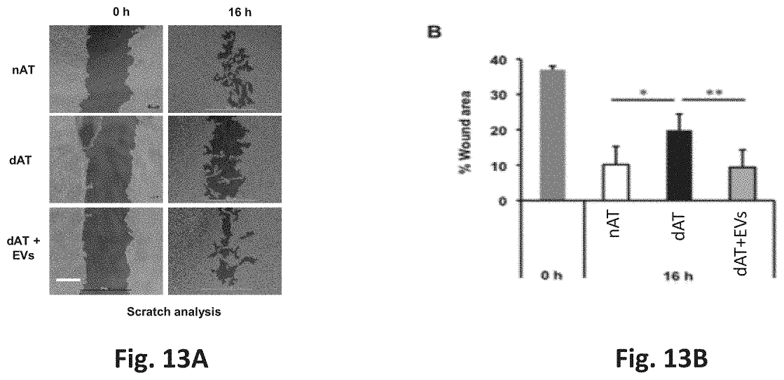

Diabetic (d) AT-MSCs were cultured with normal (n) AT-MSC-derived EVs and compared with control (dAT-MSCs) and nAT-MSCs. The results showed that the percentage of uncovered wound area after 16 hours of EV-treated dAT-MSCs was significantly decreased compared to that of untreated dAT-MSCs (wound area: EV-treated dATMSCs, 9.3%±5.0% vs. dAT-MSCs, 19.8%±4.8%, P<0.01, n=10 in each), suggesting the migration ability of dAT-MSCs was enhanced after EV internalization. Importantly, the migration ability of EV-treated dAT-MSCs was similar to that of nAT-MSCs. Representative images were captured by microscope and analyzed using the WimScratch software program (https://mywim.wimasis.com) at 0 hours and 16 hours as shown in FIG. 13A. The percentage of uncovered wound areas from nAT-MSCs, dAT-MSCs, and MV-transfected dAT-MSCs at 0 hours and 16 hours as shown in FIG. 13B. The data are the average of ten measurements from five independent wounds (mean±SD); *=P<0.05; **=P<0.01. Scale bars: 500 mm. The results show that nEVs were able to enhance the migration rate of dAT-MSCs.

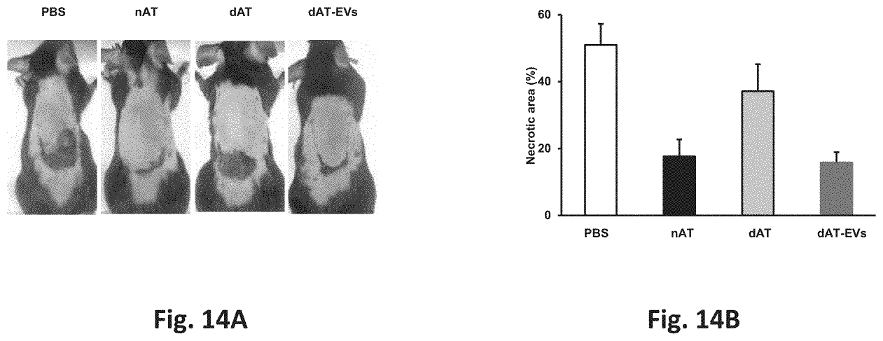

The wound healing activity of AT-MSCs was also examined using an ischemic mouse flap model in C57BL/6 mice. One week after surgery and a subcutaneous injection of AT-MSCs, the necrotic area was significantly reduced in mice injected with nEV-treated dAT-MSCs compared with mice injected with untreated dAT-MSCs (necrotic area: nEV-treated dATMSCs, 15.8%±3.0% vs. dAT-MSCs, 37.1%±8.0%, P<0.01, n=10 in each) (FIGS. 14A and 14B). Notably, the percentage of the necrotic area relative to control was not significantly different between nAT-MSC and nEV-treated dAT-MSC injected mice (necrotic area: nAT-MSCs, 17.6%±5.0% vs. nEV-treated dAT-MSCs, 15.8% 3.0%, P<0.01, n=10 in each). These findings suggest that treatment with normal MSC derived EV improved the wound healing ability of dAT-MSCs (FIGS. 14A and 14B).

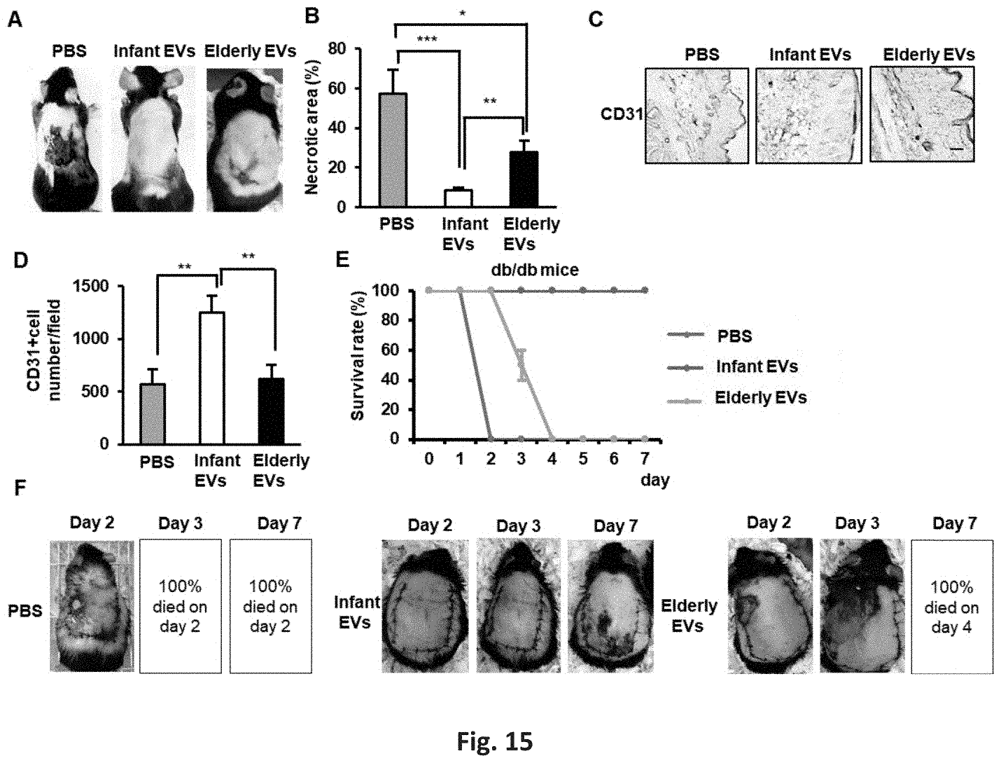

In order to suggest a functional source of EVs for cell-free therapy of the impaired neovascularization functions of the ischemic wound conditions seen in T2D subjects, the ability of iEVs and eEVs to heal wounds in type 2 diabetic (T2D) db/db mice, was evaluated. The data showed that without the injection of EVs, the mice died on day 2 post-surgery due to the severe wound, while mice injected with eEVs survived until day 3 post-surgery (FIG. 15, panel E). Of note, mice injected with iEVs survived and showed significantly improved wound healing (FIG. 15, panel F).

Collectively, these data show that iEVs possess the ability to promote the ischemic wound healing process by inducing neovascularization. Notably, these functions appear to be impaired by the effects of aging, as demonstrated by the diminished wound healing ability of eEVs compared to iEVs.

### Example 7. Activity of Wharton Jelly EVs Compared to Infant EVs

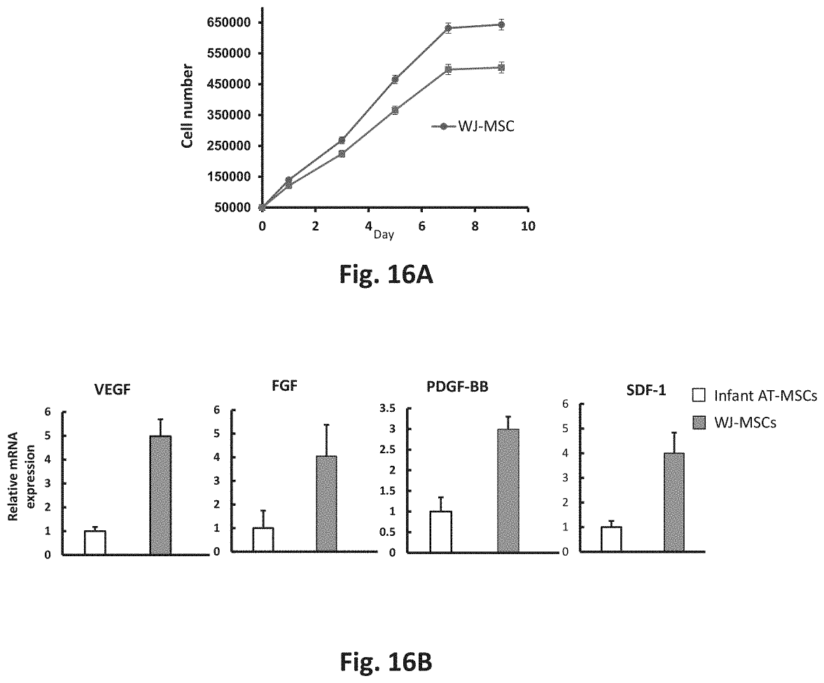

Wharton Jelly MSCs (WJ MSCs) are characterized by high proliferative ability (FIG. 16A), low cellular senescence, high immunomodulation ability and higher mRNA expression of angiogenesis related genes, including vegf, fgf, pdgf-bb, and sdf-1 than infant MSCs (FIG. 16B).

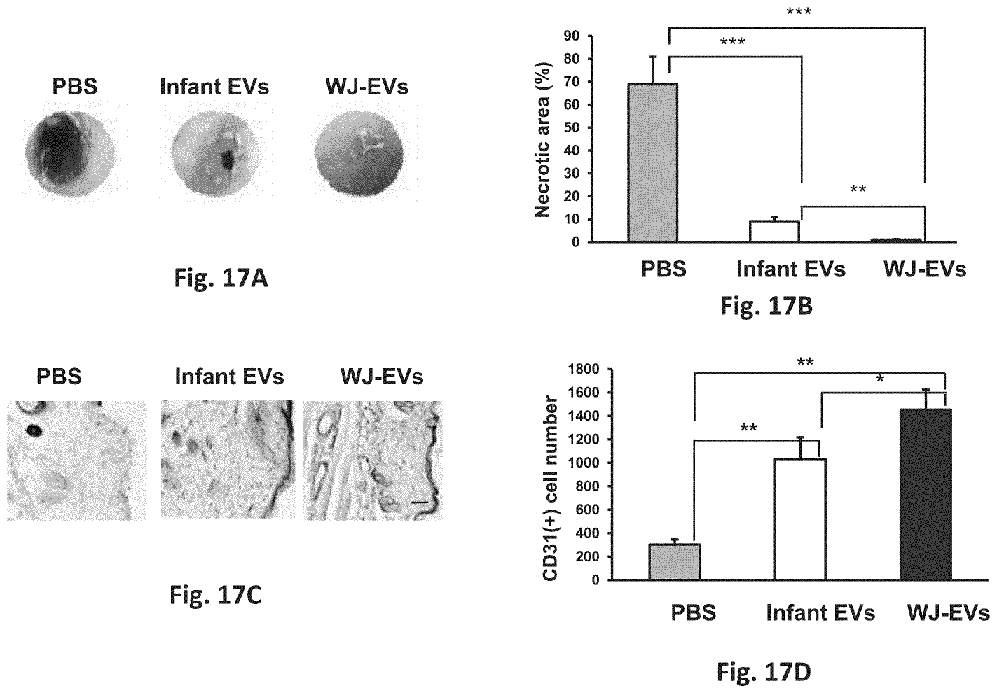

A comparison of the wound healing ability of EVs derived from WJ MSCs and those from infant MSCs in the in vivo punch wound healing model demonstrated a higher number of CD31 positive cells for WJ MSC-derived EVs and significantly greater ability to effect wound healing. (FIGS. 17A-17D). In FIGS. 17B and 17D, *=P<0.05, **=P<0.01, ***=P<0.001.

### Example 8. Separation of Exosomes and MVs in WJ Extracellular Vesicle Compositions

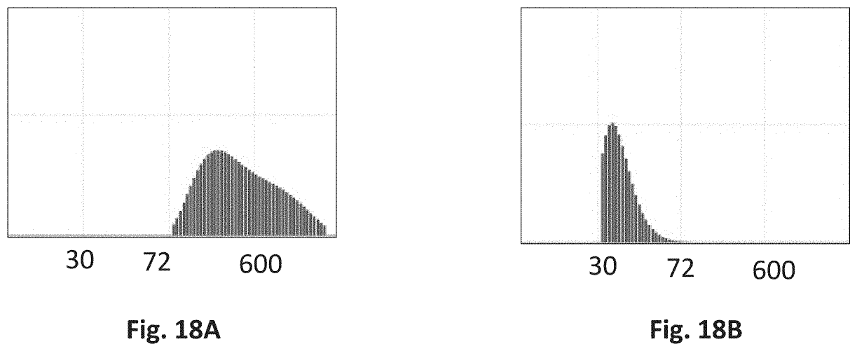

Collection of WJ-MSC-derived extracellular vesicles was carried out by seeding WJ-MSCs at 10⁶ cells/plate and culturing for 12 hours. The culture medium was replaced with fresh IMDM containing 0.25% FBS and continued culturing for an additional 48 hours. The WJ-MSC-CM (conditioned media) was collected by centrifuging at 1000 rpm for 5 minutes at 4° C. to remove dead cells. The supernatant was decanted and centrifuged at 3000 rpm for 10 minutes at 4° C. to pellet cell debris. The supernatant was decanted and centrifuged at 37,000 rpm for 35 minutes at 4° C. to pellet EVs which were analyzed using a Particle size Analyzer FDLS3000 (FIG. 18A). The composition of the pellet was predominantly microvesicles. The supernatant was decanted and centrifuged at 45,000 rpm for 70 minutes at 4° C. and the composition of the pellet was predominantly exosomes (FIG. 18B).

The MV and exosome populations were characterized by size using a Particle size Analyzer FDLS3000 to yield 107 μg of MVs per 10⁶ cells and 74 μg of exosomes per 10⁶ cells.

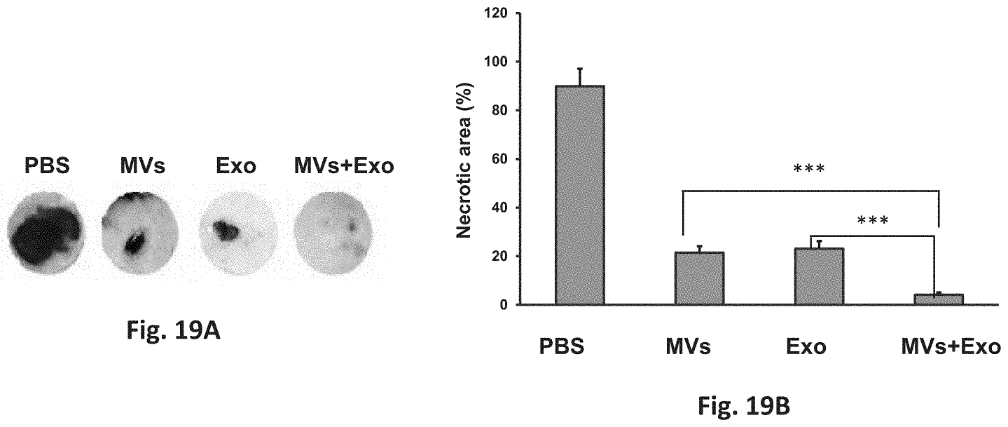

The effects of separated exosomes, MVs, and exosomes and MV mixtures were evaluated in the mouse punch biopsy model. The results showed that the fractions enriched for exosomes and MVs, respectively, exhibited comparable ability to promote wound healing. The combination of the exosomes and MV fractions in a 1:1 ratio (based on μg of protein), exhibited significantly higher wound healing potential (***=P<0.001) than either fraction alone (FIGS. 19A and 19B).

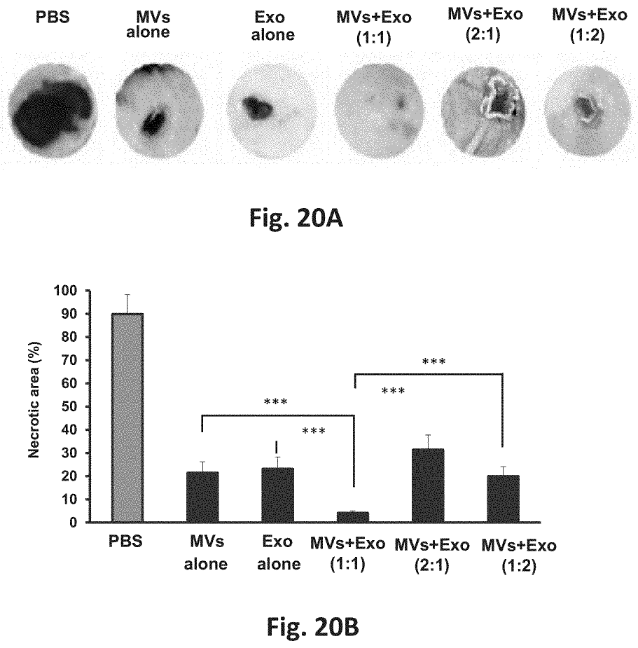

The ability of combinations of exosomes and MV to promote wound healing in the mouse punch biopsy model was further evaluated by testing the effect of exosomes alone, MVs alone, and a mixture of exosomes and MVs in a 1:1, 1:2 and 2:1 ratio with the amount of MVs or exosomes injected normalized based on μg of protein. The results shown in FIGS. 20A and B illustrate that exosome and MV mixtures with a 1:1 ratio (based on μg of protein), exhibited significantly greater wound healing potential (P<0.001) than either fraction alone. Note: A typical EV injection contained 4 μg of protein. In FIG. 20B, ***=P<0.001.

### Example 9. Dose Response of WJ-EVs to Promote Wound Healing

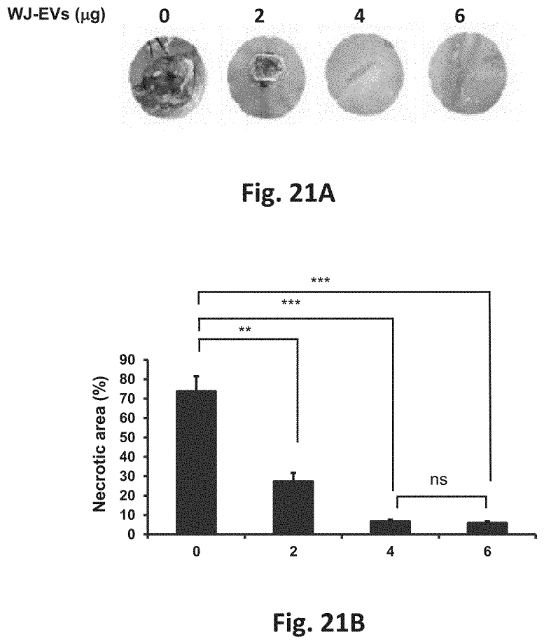

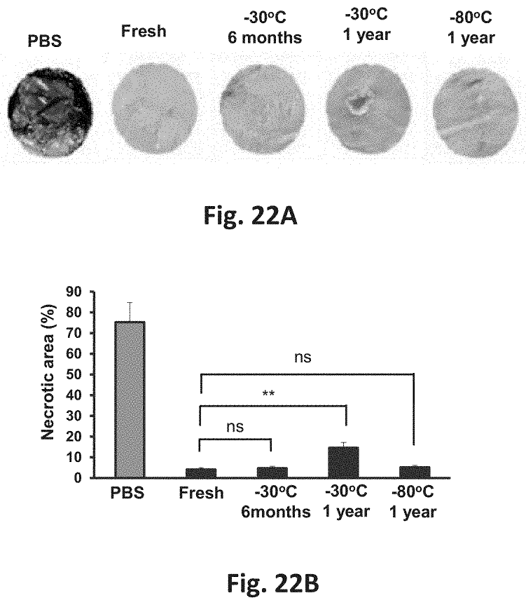

A dose response of the ability of WJ-EVs to promote wound healing in the vivo mouse punch biopsy model was evaluated by testing the wound healing ability of 0 μg, 2 μg, 4 μg, and 6 μg of WJ-EVs. The results shown in FIGS. 21A and 21B illustrate that as little as 2 μg of WJ-EVs promoted wound healing, while 4 μg and 6 μg of WJ-EVs were even better at promoting wound healing. In FIG. 21B, **=P<0.01, ***=P<0.001, and ns=not significant.

### Example 10. Storage of Infant Extracellular Vesicles

The wound healing ability of freshly isolated infant EVs and infant EVs after storage for 1 month and 3 months at −30° C. was evaluated in the mouse punch biopsy model. The ability to heal wounds in the in vivo punch wound healing model was not affected by storage for as long as 3 months at −30° C.

In a follow-up study, the wound healing ability of freshly isolated WJ-EVs in the mouse punch biopsy model after storage for 6 months and 1 year at −30° C., and for 1 year at −80° C. was evaluated. The ability to heal wounds in the in vivo punch wound healing model was not affected by storage of WJ-EVs for as long as 6 months at −30° C. and 1 year at −80° C. (FIGS. 22A and 22B). In FIG. 22B, **=P<0.01, and ns=not significant.

### Example 11. Treatment of WJ-MSCs with Edaravone Enhanced the Wound Healing Ability of WJ-EVs

WJ-MSCs at 3×10⁵ cells/plate were seeded and cultured in the presence or absence of 20 μM Edaravone until reach 80% confluency. The WJ-MSCs were passaged and seeded at 10⁶ cells/plate and culturing for 12 hours in the presence or absence of 20 μM Edaravone. The culture medium was replaced with fresh IMDM containing 0.25% FBS and continued culturing for an additional 48 hours in the presence or absence of 20 μM Edaravone. The WJ-MSC-CM was collected by centrifuging at 1000 rpm for 5 minutes, followed by centrifugation at 3000 rpm for 10 minutes at 4° C. to remove the cell debris. For WJ-MSC-EV isolation, the WJ-MSC-CM was ultracentrifuged at 37,000 rpm for 70 minutes at 4° C. The pellet was then resuspended in PBS and the protein concentration was measured using the Bradford assay.

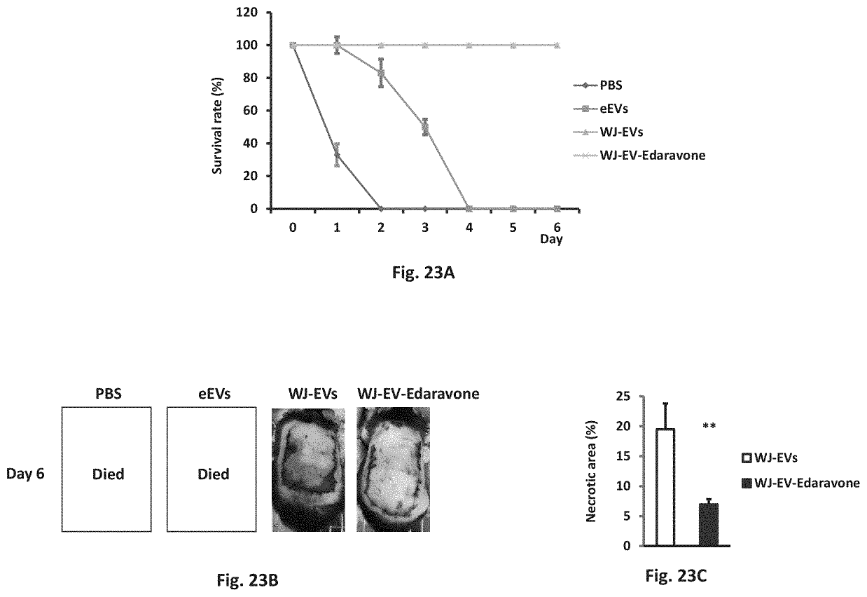

The effects of Edaravone treatment on the ability of WJ-EVs to heal wounds in type 2 diabetic (T2D) db/db mice, was evaluated. The data showed that without the injection of EVs, the mice died on day 2 post-surgery due to the severe wound, while mice injected with eEVs survived until day 3 post-surgery (FIG. 23A). Meanwhile, mice injected with WJ-EVs or Edaravone-treated-WJ-EVs survived. Of note, compared to the WJ-EV-treated mice, those injected with Edaravone-treated-WJ-EVs showed significantly improved wound healing (FIG. 23B, C) (**p<0.01).

Collectively, these data show that treatment with Edaravone in the culture of WJ-MSCs enhanced the ability of WJ-EVs to promote the ischemic wound healing process of type 2 diabetic mice.

### Example 12. Effects of WJ-MVs on Growth of Cancer Cell Lines

The effect of WJ derived EVs on growth of cancer cell lines was evaluated by co-culture of EVs with human breast cancer, human cervical carcinoma, and human promyelocytic leukemia cancer cell lines. WJ-MSCs were obtained using the procedure described in Example 1. Collection of WJ-MSC-derived EVs was carried out by seeding WJ-MSCs at 10⁶ cells/plate and culturing for 12 hours. The culture medium was replaced with fresh IMDM containing 0.25% FBS and continued culturing for an additional 48 hours. The WJ-MSC-CM (conditioned media) was collected by centrifuging at 1000 rpm for 5 minutes to remove dead cells. The supernatant was decanted and centrifuged at 3000 rpm for 10 minutes to pellet cell debris. The supernatant was decanted and ultracentrifuged at 37,000 rpm for 70 minutes at 4° C. The pellet was then resuspended in PBS and the protein concentration was measured using the Bradford assay.

5×10⁴ cells from a variety of cancer cells lines were seeded in 35 mm cell culture dishes and cultured for 8 hours following by adding a 5 ng total protein of WJ-MSC-derived EVs. The culture medium was changed every 3 days followed by adding 5 ng total protein of WJ-MSC-derived EVs. The growth of the cancer cells lines was observed 12 days following initiation of EV incorporation. The effects of EVs derived from iAT-MSC and eAT-MSC were also evaluated. Cancer cells without incorporation of EVs were used as the control. The results of the study are shown in Table 1, below.

TABLE 1
Effects of WJ-MVs on growth of cancer cell lines.

| Cancer type | Cell line | WJ-EVs | iAT-EVs | eAT-EVs |
|---|---|---|---|---|
| Human | MCF-7 | Impaired | Impaired | Promoted |
| breast | | proliferation | proliferation | proliferation |
| cancer | | Induced | Induced | Impaired |
| | | apoptosis | apoptosis | apoptosis |
| | MDA- | Impaired | Impaired | Promoted |
| | MB-231 | proliferation | proliferation | proliferation |
| | | Induce | Induce | Impaired |
| | | apoptosis | apoptosis | apoptosis |
| Human | Hela | Impaired | Not | N.D. |
| cervical | | proliferation | determined | |
| carcinoma | | Induce | (N.D.) | |
| | | apoptosis | | |
| Human | HL60 | No effects | N.D. | N.D. |
| promyelocytic | | | | |
| leukemia | | | | |

### Example 13. Effect of SARS COV2 Spike Protein Peptides on Human Lung Epithelial Cells

### Material and Methods for Examples 13-16

### MSC Isolation and Culture

Human adipose tissue was obtained from non-diabetic donors (n=4, male, average age of 70), who were undergoing procedures in the Department of Cardiovascular Surgery, University of Tsukuba Hospital, Tsukuba, Japan. The human adipose tissues were rinsed with phosphate-buffered saline (PBS) and minced with scissors and a scalpel into pieces with a size of less than 3 mm. The pieces were treated with 0.1% collagenase (Nitta Gelatin, Osaka, Japan) in PBS and 20% fetal bovine serum (FBS; Hyclon, South Logan, Utah) for 45 minutes at 37° C., then filtered through a 100-mm nylon mesh (BD Biosciences, San Jose, Calif.). After incubation, samples were centrifuged to remove the collagenase solution. The isolated cells were cultured in MSC culture medium containing Iscove's modified Dulbecco's medium (IMDM) (Invitrogen, Carlsbad, Calif.) containing 10% FBS, 2 mg/mL I-glutamine (Invitrogen), 5 ng/mL human basic-FGF (Peprotech, London, United Kingdom), and 0.1% (v/v) penicillin-streptomycin (100 U/mL penicillin, 0.1 mg/mL streptomycin; Invitrogen) at 37° C., 5% CO2 in a humidified atmosphere. The cells are referred to as normal adipose tissue MSCs (nAT-MSCs).

Once the appearance of isolated nAT-MSCs was confirmed, medium containing non-adherent cells was removed and replaced with fresh medium. Frozen cell stocks of nAT-MSCs were prepared using Cell Banker solution (ZENOAQ) and stored in liquid nitrogen for future experiments. All MSCs used for the experiments described in the examples were at passage 4-8.

Human umbilical cord tissue was obtained from non-diabetic donors (n=4, female, average age at 32), who were undergoing cesarean section in the Department of Obstetrics and Gynecology, University of Tsukuba Hospital, Tsukuba, Japan. Human umbilical cord tissue was rinsed with cold phosphate-buffered saline (PBS) to remove blood clots and cut to expose the blood vessels and Wharton's Jelly. The blood vessels were removed, and Wharton's Jelly was collected and cut into 1-2 mm pieces. After that, the Wharton's Jelly pieces were incubated with trypsin solution at solution at 37° C. for 30 minutes in an incubator under 5% CO₂ for partial digestion. After incubation, an equal volume of MSC culture medium was added and incubated for 3 minutes then 15-20 digested tissue pieces were carefully plated on a culture dish containing MSC culture medium and were cultured in at 37° C. in a 5% CO₂ incubator with a humidified atmosphere for 3 days. These cells are referred to as normal Wharton Jelly MSCs (nWJ-MSCs).

Once the appearance of isolated WJ-MSCs was confirmed, medium containing non-adherent cells was removed and replaced with fresh medium. Frozen cell stocks of WJ-MSCs were prepared using Cell Banker solution (ZENOAQ) and stored in liquid nitrogen for future experiments. All MSCs used for the experiments described in the examples were at passage 4-8.

### Human Lung Epithelial Cell Culture

The Calu-3 cell line (LGC Standards, Cat No. ATCC-HTB-55, American Type Culture Collection-ATCC,) was kindly provided by Department of Human Pathology, University of Tsukuba. Calu-3 cells were cultured in Eagle's Minimum Essential Medium (EMEM) medium containing 10% FBS, 1% Penicillin/Streptomycin at 37° C. in a 5% CO₂ incubator with a humidified atmosphere. The media was changed 3 times per week. When reaching 80% confluency, the cells were harvested and subcultivated at a ratio of 1:5.

### Proliferation Assay

Calu-3 cells were seeded at 10⁵ cells/well in 24-well plates in EMEM medium containing 10% FBS with 1% Penicillin/Streptomycin and cultured at 37° C. in a 5% CO₂ incubator with a humidified atmosphere. The cells were harvested, and the number of live cells were counted daily after staining with Trypan blue solution (Nacalai Tesque, Kyoto, Japan) using a hemocytometer.

### Induction of Human Lung Epithelial Cells by SARS COV2 Spike Protein Peptides

A number of the wells in 24-well plates that were seeded with 5×10⁵ Calu-3 cells were exposed to SARS COV2 spike protein peptides (SARS-CoV-2 pepTivator Peptide Pools Prot_S; Miltenyi Biotech) at 30 pmol for 24 hours before collecting for the further analysis.

Gene Expression Analysis (qPCR)

To examine gene expression, total RNA was collected from Calu-3 cells and isolated by Sepasol-RNA I Super G (Nacalai Tesque, Lot. L0A1475) following the protocol of the manufacturer. Total RNA (1 μg) was reverse transcribed using an RT-PCR kit (TOYOBO, Japan). cDNA was analyzed using a GeneAmp 7500Fast Real-Time PCR System (Applied Biosystems) using SYBR green reagent (TOYOBO). The expression levels of the target genes were analyzed using the ΔΔCt method. The sequences of the primer sets used for the PCR reactions are shown in Table 1.

TABLE 1
Primer sets used for quantitative PCR

| Gene | Forward primer | Reverse primer |
|---|---|---|
| CCL17 | ACTGTCTCCCGGGACTACCT | TTTAATCTGGGCCCTTTGTG |
| TNFa | TCCTTCAGACACCCTCAACC | AGGCCCCAGTTTGAATTCTT |
| IL6 | ACAAGAGTAACATGTGTGAA | TATACCTCAAACTCCAAAAG |
| | AGCAG | ACCAG |
| IFNβ | CATTACCTGAAGGCCAAGGA | CAGCATCTGCTGGTTGAAGA |
| IFNλ3 | CTGCTGAAGGACTGCAAGTG | GAGGATATGGTGCAGGGTGT |
| IP10 | AGGAACCTCCAGTCTCAGCA | CAAAATTGGCTTGCAGGAAT |
| CXCL9 | CCACCGAGATCCTTATCGAA | GCTAACTGGGCACCAATCAT |
| β-actin | GTGCGTGACATTAAGGAGAA | GTACTTGCGCTCAGGAGGAG |
| | GCTGTGC | CAATGAT |

### Co-Culture of MSCs and Calu-3 Cells

Calu-3 cells were seeded at 5×10⁵ cells in the lower chamber of an 8-μm pore transwell (Corning Incorporated, New York, N.Y., USA) containing a total of 500 μL. Cells were maintained at 37° C. in a 5% CO₂ atmosphere for 6 hours to allow for cell attachment, then exposed to SARS COV2 spike protein peptides for 24 hours. After that, 10⁵ MSCs were seeded into the upper chamber of the transwell and coculture was maintained at 37° C. in a 5% CO₂ atmosphere for an additional 24 hours. At the end of the co-culture period, Calu-3 cells were collected, and genetic analysis was performed.

### EV Isolation

The medium of subconfluent cultures of nAT-MSCs or nWJ-MSCs was changed to Iscove's modified Dulbecco's medium (IMDM) containing 1% Penicillin/Streptomycin. After 24 hours, the supernatant was collected and centrifuged at 1000×g for 5 minutes to remove cells from medium, then centrifuged at 2100×g for 20 minutes to remove cell debris from medium. Cell-free supernatants were ultracentrifuged at 100,000×g for 70 minutes at 4° C. The pellets were stained with PKH26-red (PKH26 linker, SIGMA, Lot.MKCG0926) for 5 minutes at room temperature, then PBS with 0.2% FBS (SIGMA, Lot.BCBE1122) was added to stop the staining reaction. The ultracentrifuge step was repeated a second time under the same conditions to yield pellets of EVs. The EV pellets were collected, then characterized by quantification of the protein concentration using the Bradford method (BioRad, CA, USA), size measurement using a Particle Size Analyzer (FDLS3000; Shimadzu Corporation, Kyoto, Japan), and morphology analysis using a Transmission Electron Microscopy (JEM-1400Flash, JEOL Ltd., Tokyo, Japan).

Treatment of Calu-3 with High Glucose Concentration

Samples of 5×10⁵ Calu-3 cells were cultured in medium containing EMEM with 10% FBS and 1% Penicillin/Streptomycin as well as 10 mM, 20 mM, or 30 mM glucose. The phenotype and gene analysis of Calu-3 cells were examined after 24, 48, and 72 hours. Calu-3 cells cultured in medium without glucose was used as the control.

### Statistical Analyses

Data were statistically analyzed by the Mann Whitney U-test using the GraphPad Prism 5 software program (GraphPad Software, CA, USA). Data are presented as the mean±standard deviation. P<0.05 was considered as significant.

The effect of EVs (microvesicles and exosomes) from MSCs on the regulation of pro-inflammatory cytokines in Calu-3 human lung epithelial cells induced by SARS COV2 spike protein peptides was evaluated.

### Experimental

In order to examine the response of human lung epithelial cells to SARS-Cov2 peptides, Calu-3 human lung adenocarcinoma cells were exposed to SARS-CoV-2 pepTivator Peptide Pools Prot_S (Miltenyi Biotech), a spike glycoprotein (“Prot_S” or “SARS COV2 spike protein peptides”) and the expression of CCL17 (a cytokine the concentration of which in blood is decreased below the standard value in humans in the early stage of SARS-CoV-2), and pro-inflammatory cytokines. SARS-CoV-2 spike glycoprotein is known to be responsible for the recognition and binding of SARS-CoV-2 to host cells.

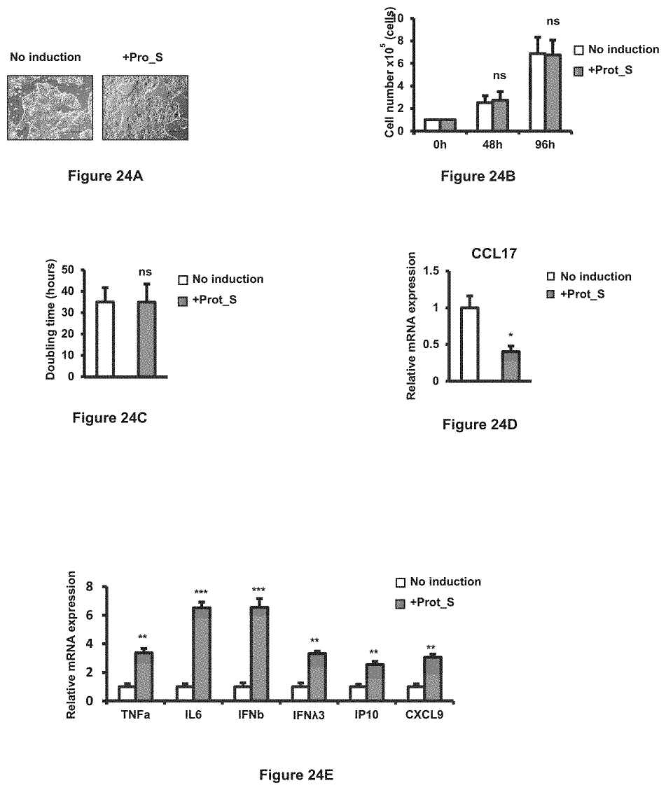

5×10⁵ human lung epithelial cells were exposed to 30 pmol Prot_S for 24 hours. The results show that exposure to Prot_S did not alter the morphology of Calu-3 cells, which maintained their epithelial morphology after 96 hours of exposure to Prot_S (FIG. 24A). In addition, exposure to Prot_S did not affect the proliferation rate or doubling time of Calu-3 cells, which have an average doubling time of approximately 33 hours (FIGS. 24B and 24C). An analysis of relative mRNA expression in Calu-3 cells 24 hours after exposure to Prot_S was conducted by qPCR as described above under the section entitled “Gene Expression Analysis”. The results demonstrated decreased expression of CCL17 (FIG. 24C), and increased expression of the pro-inflammatory cytokines TNFα, IL6, IFN1β, IFNγ3, IP-10, and CXCL9 (FIG. 24D).

Taken together, these data show upregulation of pro-inflammatory cytokines and downregulation of CCL17 in human lung epithelial cells upon exposure to SARS COV2 spike protein peptides.

### Example 14. EVs Derived from MSCs Reduced Expression of Pro-Inflammatory Cytokines Upregulated by SARS COV2 Spike Protein Peptides in Human Lung Epithelial Cells

A. The effects of MSCs from healthy (normal) donor adipose tissue (nAT-MSCs) and healthy (normal) donor Wharton's Jelly (nWJ-MSCs) on inflammatory cytokine expression in human lung epithelial cells after exposure to (SARS COV2 spike protein peptides) was evaluated using a transwell coculture system.

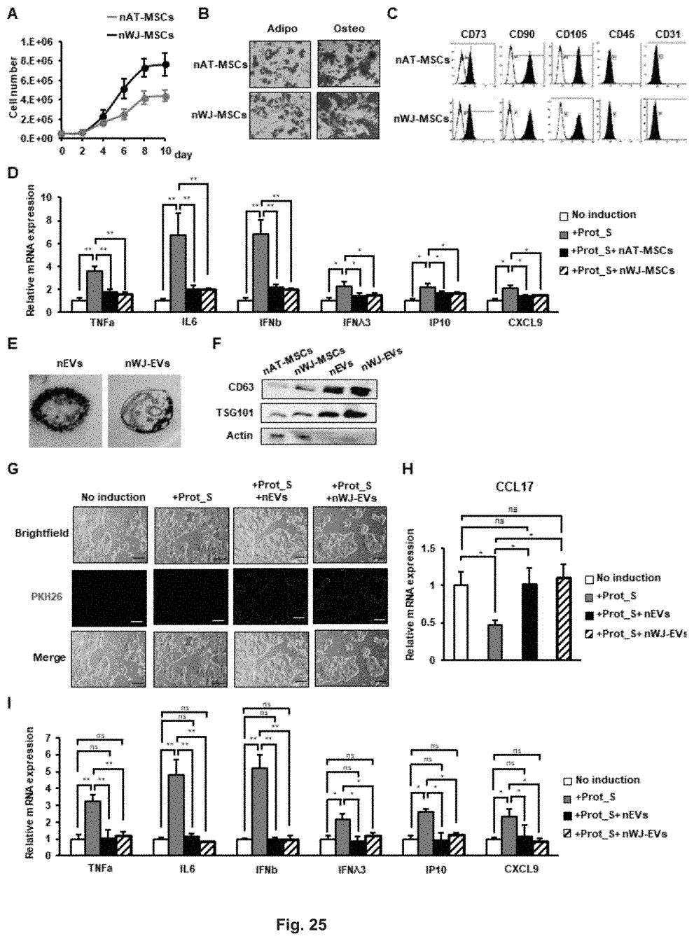

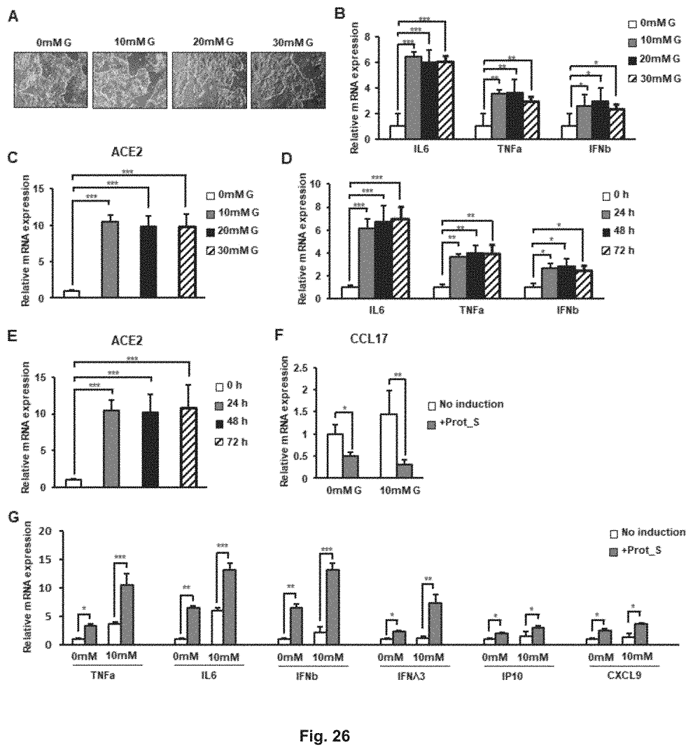

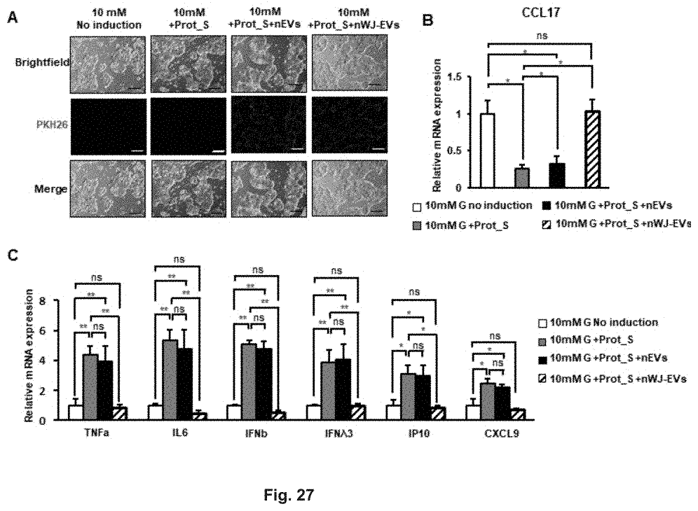

nAT-MSCs and nWJ-MSCs were characterized in terms of proliferation rate (FIG. 25, panel A), the ability to differentiate to adipocytes and osteocytes (FIG. 25, panel B), and the expression of MSCs markers CD73, CD90, and CD105 (positive), and CD45 and CD31 (negative) (FIG. 25, panel C).

5×10⁵ human Calu-3 human lung epithelial cells were exposed to 30 pmol Prot_S (Miltenyi Biotech) for 24 hours using a transwell coculture system. After 24-hour exposure to Prot_S, Calu-3 cells were co-cultured with MSCs for an additional 24 hours as described above.

At the end of the co-culture period, Calu-3 cells were collected, and genetic analysis was performed. An analysis of inflammatory cytokine expression showed that co-culture with either nAT-MSCs or nWJ-MSCs reduced the expression of pro-inflammatory cytokines in Calu-3 cells that was evident after exposure to Prot-S (FIG. 25, panel D).

Given that studies have shown that the functions of MSCs may be mediated by EVs, a follow-up study was conducted to compare the effect of EVs to the observed effects of MSCs on expression of pro-inflammatory cytokines in Calu-3 cells after exposure to Prot-S.

In this study 5×10⁵ human Calu-3 human lung epithelial cells were exposed to 30 pmol Prot_S (Miltenyi Biotech) for 24 hours using a transwell coculture system. After 24-hour exposure to Prot_S, 100 μg nEVs-labeled by PKH-26-red or 100 μg nWJ-EVs-labeled by PKH-26-red were added to the Calu-3 cells for another 24 hours.

Analysis of EVs under a transmission electron microscopy showed that both nEVs and nWJ-EVs exhibited characteristic round morphology with a diameter from 60-200 nm (FIG. 2E). In addition, both nEVs and nWJ-EVs expressed the EVs markers CD63 and TSG101 and were negative for expression of actin (FIG. 25, panel F).

nEVs and nWJ-EVs respectively were incorporated in Calu-3 cells by co-culture of 5×10⁵ Calu-3 cells with 100 μg of EVs (containing a ratio of exosomes to MVs of approximately 1:1.5) for an additional 24 hours. Incorporation of nEVs or nWJ-EVs into Calu-3 cells was confirmed by detection of the PKH-26-red signal viewed with fluorescence microscopy 24 hours following the initiation of co-culture (FIG. 25, panel G).

As the results show, the incorporation of nEVs or nWJ-EVs into Calu-3 cells increased the expression of CCL17 which was decreased by exposure to Prot_S (FIG. 25, panel H). In addition, the incorporation of nEVs or nWJ-EVs into Calu-3 cells decreased the expression of pro-inflammatory cytokines (TNFα, IL6, IFN1β, IFNγ3, IP-10, and CXCL9) to the (baseline) expression level observed prior to exposure to Prot_S (FIG. 25, panel I).

Taken together, these data suggest that nEVs and nWJ-EVs can reduce the SARS COV2 spike protein peptide-induced overexpression of pro-inflammatory cytokines and recover the SARS COV2 spike protein peptide induced decrease in expression of CCL17 in Calu-3 cells.

### Example 15. High Glucose Concentration Induced Expression of ACE2 Receptor in Human Lung Epithelial Cells and Response to SARS COV2 Spike Protein Peptides in the Presence of High Concentrations of Glucose

In the study detailed below, the effects of exposure to a high concentration of glucose on human lung epithelial cells (Calu-3) was evaluated together with the response of Calu-3 cells cultured in media containing a high concentration of glucose and exposed to Prot_S (Miltenyi Biotech) SARS COV2 spike protein peptides.

5×10⁵ human Calu-3 human lung epithelial cells were cultured in media including glucose at a concentration of 0 mM, 10 mM, 20 mM or 30 mM. Culture in the presence of such high concentrations of glucose for 24 hours did not affect the morphology of Calu-3 cells (FIG. 26, panel A). However, after 24 hours exposure to glucose concentrations of 10 mM, 20 mM and 30 mM, Calu-3 cells exhibited an increase in the expression of pro-inflammatory cytokines (IL-6, TNFα and IFN-β) (FIG. 26, panel B), and an increase in the expression of ACE2 (a receptor for the infection of SARS-CoV-2 (FIG. 3C). The increase in expression of pro-inflammatory cytokines (IL-6, TNFα and IFN-β) and ACE2 in Calu-3 cultured in 10 mM, or 20 mM, or 30 mM glucose was substantially the same, so 10 mM glucose was used for further studies (FIG. 26, panel D and FIG. 26, panel E).

A follow-up study was conducted to evaluate the effect of exposure of Calu-3 human lung epithelial cells to glucose concentrations of 10 mM, 20 mM and 30 mM for longer than 24 hours. In this study, 5×10⁵ human Calu-3 cells were cultured in media containing 10 mM glucose for 24, 48, or 72 hours, then analyzed for the expression of pro-inflammatory cytokines IL-6, TNFα and IFN-β and ACE2. The results show that there was no further increase in expression of pro-inflammatory cytokine or ACE, respectively, in Calu-3 cells exposed to high glucose concentrations for longer than 24 hours (FIGS. 3D and 3E).

In a further study, the effect of Prot_S (SARS COV2 spike protein peptides) on human Calu-3 cells cultured in medium containing 10 mM glucose was evaluated. 5×10⁵ human Calu-3 human lung epithelial cells were cultured in media containing 10 mM glucose for 24 hours, followed by exposure to 30 pmol Prot_S for an additional 24 hours. The results show that Calu-3 cells cultured in the presence of 10 mM glucose and exposed to Prot_S exhibited lower expression of CCL17 (FIG. 26, panel F) and higher expression of pro-inflammatory cytokines (TNFα, IL6, IFN1β, IFNγ3, IP-10, and CXCL9) (FIG. 26, panel G), than Calu-3 cells exposed to Prot_S in the absence of 24-hour pre-treatment with a high concentration of glucose.

Taken together, these data demonstrate that pre-exposure of Calu-3 lung epithelial cells to a high glucose concentration induces (a) expression of ACE2, (b) decreased expression of CCL17 and (c) increased expression of pro-inflammatory cytokines in response to SARS COV2 spike protein peptides.

### Example 16. Evaluation of the Effect of nEVs and nWJ-EVs on Human Lung Epithelial Cells Exposed to SARS COV2 Spike Protein Peptides in the Presence of High Concentrations of Glucose

Given the role of diabetes in the severity of COVID-19 disease, the pro-inflammatory cytokine response of lung epithelial cells to SARS COV2 spike protein peptides (Prot_S) was evaluated under high glucose concentrations. In addition, the effect of MSC-derived EVs (nEVs and nWJ-EVs) on the phenotype (and recovery) of lung epithelial cells exposed to Prot_S under high glucose concentration was tested.

5×10⁵ Calu-3 cells were cultured in the presence of 10 mM glucose for 24 hours, exposed to 30 pmol Prot_S (SARS COV2 spike protein peptides) for 24 hours, then co-cultured with 100 μg of EVs (nEVs or nWJ-EVs with a ratio of exosomes to MVs of approximately 1:1.5) for an additional 24 hours.

Incorporation of nEVs or nWJ-EVs into Calu-3 cells was confirmed by detection of the PKH-26-red signal viewed with fluorescence microscopy 24 hours following the initiation of co-culture (FIG. 27, panel A).

The results showed that Calu-3 cells cultured under high glucose concentration and exposed to Prot_S with nEVs incorporated into the Calu-3 cells compared to Calu-3 cells which lacked nEV incorporation exhibited no significant difference in the expression of CCL17 (FIG. 27, panel B), and no reduction in Prot_S-induced overexpression of pro-inflammatory cytokines (TNFα, IL6, IFN1β, IFNγ3, IP-10, and CXCL9) (FIG. 27, panel C).

In contrast, the results showed that Calu-3 cells cultured under high glucose concentration and exposed to Prot_S with nWJ-EVs incorporated into the Calu-3 cells exhibited increased expression of CCL17 to a level comparable to the (baseline) CCL17 expression level detected prior to exposure to Prot_S (FIG. 27, panel B), and decreased the expression of pro-inflammatory cytokines (TNFα, IL6, IFN1β, IFNγ3, IP-10, and CXCL9) to a level comparable to the (baseline) expression level observed prior to exposure of the Calu-3 human lung epithelial cells to Prot_S (FIG. 27, panel C).

Taken together, these data show a difference in function of nEVs and nWJ-EVs on Calu-3 cells cultured under a high glucose concentration and exposed to Prot_S.

Of note, while treatment with EVs from adipose tissues-derived MSCs (nEVs) and Wharton's Jelly-derived MSCs (nWJ-EVs) both reduced the decreased expression of CCL-17 and increased expression of pro-inflammatory cytokines in Prot_S-induced Calu-3, only nWJ-EVs showed the ability to recover the response of Calu-3 to Prot_S under high glucose concentration.

The studies described herein demonstrate that the expression of pro-inflammatory cytokines is increased and the expression of CCL17 is decreased in human lung epithelial cells exposed to SARS COV2 spike protein peptides (SARS-CoV-2 pepTivator Peptide Pools Prot_S), SARS COV2 spike protein is a glycoprotein which is responsible for the recognition and binding of SARS-CoV-2 to host cells.

An increase in expression of pro-inflammatory cytokines such as TNFα, IL6, IFN1β, IFNγ3, IP-10, and CXCL9 is indicative of a cytokine storm. See, e.g., Sugiyama et al. (2021) Gene, Vol. 766, 145145; Dell Valle, D. (2020) Nature Medicine Vol. 26, 1636-1643; and de la Rica, R. (2020) Frontiers in Immunology 558898.

The disclosure is based, in part, on the surprising finding that treatment of lung cells with exosomes and microvesicles derived from mesenchymal stem cells decrease the SARS COV2 spike protein peptide-induced expression of pro-inflammatory cytokines and increase the expression of CCL17 in human lung epithelial cells.

The studies described herein also demonstrated that exposure to a high glucose concentration induces the expression of ACE2, a further increase in the expression of pro-inflammatory cytokines, and a further decrease in the expression of CCL17 in human lung epithelial cells exposed to SARS COV2 spike protein peptides.

The disclosure is further based on the surprising finding that WJ-derived exosomes and microvesicles recovered (a) (reduced) the expression of pro-inflammatory cytokines, and (b) (increased) the expression of CCL17, that were increased (pro-inflammatory cytokines) or decreased (CCL17) peptides in human lung epithelial cells exposed to a high glucose concentration and exposed to SARS COV2 spike protein.

Taken together, these data show the potential efficacy in use of EV compositions (comprising a mixture of microvesicles and exosomes) in the treatment of certain disorders of the lungs, including but not limited COVID-19, over-reactive inflammatory responses, cytokine storms, and/or ARDS.

What is claimed is:

1. An extracellular vesicle (EV) composition derived from mesenchymal stem cells (MSCs) and comprising a mixture of microvesicles (MVs) and exosomes, wherein the composition is effective to decrease the time for skin wound healing relative to a composition of microvesicles or exosomes alone.

2. The EV composition according to claim 1, wherein the MSCs are derived from an infant.

3. The EV composition according to claim 1, wherein the MSCs are derived from Wharton's Jelly.

4. The EV composition according to claim 1, wherein the EV composition comprises exosomes with a diameter of less than 100 nm.

5. The EV composition according to claim 1, wherein the EV composition comprises microvesicles (MVs) with a diameter of from about 100 nm to about 1000 nm.

6. The EV composition according to claim 1, wherein the EV composition comprises exosomes and microvesicles that express CD63 and/or TSG101 membrane proteins.

7. The EV composition according to claim 1, wherein the EV composition is derived from MSCs treated with Edaravone.

8. The EV composition according to claim 1, wherein the EV composition comprises exosomes and MVs combined in a ratio of from about 0.5 to 1.5.

9. The EV composition according to claim 8, wherein the EV composition comprises exosomes and MVs combined in a ratio of about 1:1.

10. A method of treating a necrotic skin wound, comprising application of an EV composition according to claim 1 to the skin wound, wherein the necrotic area associated with the skin wound is decreased by at least 10%, at least 20%, at least 30%, at least 40%, at least 50%, at least 60%, at least 70%, or at least 80% or more within five to fourteen days following treatment.

11. The method according to claim 10, wherein the skin wound is from a patient with Type 2 diabetes.

12. The method according to claim 1, wherein the skin wound is a pressure wound.

13. The method according to claim 10, wherein the skin wound is a diabetic ulcer.

14. The method according to claim 10, wherein the EV composition is applied topically.

15. The method of according to claim 10, wherein the EV composition is injected.

16. A method of reducing pro-inflammatory cytokine expression in lung cells that was increased following contact with SARS-CoV-2, comprising administering to the lung cells an EV composition derived from human mesenchymal stem cells (MSCs) and comprising exosomes and MVs, wherein following said administration, pro-inflammatory cytokine expression in the lung cells is significantly reduced.

17. The method according to claim 16, wherein the pro-inflammatory cytokines include one or more of TNFα, IL6, IFN1β, IFNγ3, IP-10, and CXCL9.

18. A method of reversing the reduction in CCL17 expression in lung cells that was increased following contact with SARS-CoV-2, comprising administering to the lung cells an EV composition derived from human mesenchymal stem cells (MSCs) and comprising exosomes and MVs, wherein following said administration the CCL17 expression in lung cells is increased to substantially the same level detected prior to contact with SARS-CoV-2.

19. The method according to according to claim 16, wherein the human MSCs are derived from Wharton's Jelly.

20. The method according to according to claim 16, wherein the EV composition comprises exosomes with a diameter of less than 100 nm.

21. The method according to according to claim 16, wherein the EV composition comprises microvesicles (MVs) with a diameter of from about 100 nm to about 1000 nm.

22. The method according to according to claim 16, wherein the EV composition comprises exosomes and microvesicles that express CD63 and/or TSG101 membrane proteins.

23. The method according to claim 16, wherein the EV composition comprises exosomes and microvesicles in a ratio of about 1 to 1.5.

24. The method according to claim 16, wherein the lung cells were exposed to a high concentration of glucose prior to exposure to SARS-CoV-2.

25. The method according to claim 24, wherein the lung cells were derived from a patient with Type 2 diabetes.
